(19) United States
(12) Patent Application Publication
(10) Pub. No.: US 2023/0352134 A1
(43) Pub. Date: Nov. 2, 2023

(54) AI BASED SYSTEMS AND METHODS FOR PROVIDING A CARE PLAN

(71) Applicant: CVS Pharmacy, Inc., Woonsocket, RI (US)

(72) Inventors: Sheikh Sadid Al Hasan, Weymouth, MA (US); Venkata Rama Bh Bachimanchi, Rocky Hill, CT (US); Dionyssios Mintzopoulos, Boston, MA (US); Rahul Bhasin, Woonsocket, RI (US); Vikram Bundela, Hartford, CT (US)

(21) Appl. No.: 18/128,592

(22) Filed: Mar. 30, 2023

Related U.S. Application Data
(60) Provisional application No. 63/327,087, filed on Apr. 4, 2022.

(51) Int. Cl. G16H 10/60; G16H 50/30; G16H 20/00

(52) U.S. Cl. CPC G16H 20/00; G16H 10/60; G16H 50/30

(57) ABSTRACT

A method is provided that includes: providing data inputs to a machine learning model, where the data inputs include electronic patient data obtained from electronic records describing a health history of the patient. The method includes receiving an output from the machine learning model, where the output is generated based on the machine learning model processing the data inputs and includes identified care gaps for the patient. The method includes determining a treatment effect for each of the identified care gaps and assigning a treatment effect score to each of the identified care gaps. The method includes prioritizing the identified care gaps based on the treatment effect score assigned thereto and, based on the prioritization, determining one or more recommended patient actions for the patient. The method includes generating and transmitting an electronic communication that describes the one or more recommended patient actions for the patient.

### CROSS REFERENCE TO RELATED APPLICATIONS

The present application claims the benefit of and priority to U.S. Provisional Application No. 63/327,087, filed on Apr. 4, 2022, entitled “AI BASED SYSTEMS AND METHODS FOR PROVIDING A CARE PLAN,” which application is incorporated herein by reference in its entirety.

### FIELD OF THE DISCLOSURE

The disclosure relates to systems and methods for providing a care plan (e.g., a healthcare plan) for a patient, and particularly, for AI based systems and methods for providing care recommendations for the patient based on a modeling analysis of a health history, gaps in care, and social determinants of health of the patient.

### BACKGROUND

Healthcare providers and other entities involved in the delivery of treatments to members are able to observe medical histories of their members. Healthcare providers routinely access and review healthcare data (e.g., treatment histories) of patients under their care to generate healthcare plans for the patients. Improved techniques for generating healthcare plans for patients are desired.

### SUMMARY

A method of automatically generating a patient care plan includes: providing data inputs to a machine learning model, where the data inputs include electronic patient data obtained from a plurality of electronic records describing a health history of the patient; receiving an output from the machine learning model, where the output received from the machine learning model is generated based on the machine learning model processing the data inputs and includes a plurality of identified care gaps for the patient; determining a treatment effect for each of the plurality of identified care gaps; based on the treatment effect determined for each of the plurality of identified care gaps, assigning a treatment effect score to each of the plurality of identified care gaps; prioritizing the plurality of identified care gaps based on the treatment effect score assigned thereto; based on the prioritization of the plurality of identified care gaps, determining one or more recommended patient actions for the patient; generating an electronic communication that describes the one or more recommended patient actions for the patient; and transmitting the electronic communication via a communication network to a communication device.

In some aspects, the electronic communication is transmitted to a communication device of the patient.

In some aspects, the electronic communication is transmitted to a communication device of a care manager of the patient.

In some aspects, the one or more recommended patient actions are determined, at least in part, with reference to a patient library that includes a plurality of electronic patient data records with care gap information and information describing a success of closing a care gap with an action.

In some aspects, the information describing the success of closing the care gap with the action includes a count of a number of patient admissions to a healthcare facility following an identification of the care gap.

In some aspects, the treatment effect score for each identified care gap is based, at least in part, on a prediction of success associated with closing each identified care gap.

In some aspects, the electronic communication is transmitted via a selected communication channel.

In some aspects, the selected communication channel is selected based on a probability of closing a care gap having the highest treatment effect score and where the selected communication channel includes at least one of email, direct mail, SMS, and an automated outbound calling campaign.

In some aspects, the electronic patient data includes claims-based electronic data.

In some aspects, the electronic patient data further includes electronic medical record (EMR) data.

In some aspects, the claims-based electronic data includes data describing at least one insurance medical and/or insurance claim made by at least one of the patient and a healthcare provider of the patient.

In some aspects, the electronic patient data includes device data obtained from at least one device associated with the patient.

Some examples include: receiving clinician feedback for the one or more recommended patient actions; providing the clinician feedback as training data to the machine learning model; and updating at least one coefficient of the machine learning model based on providing the training data to the machine learning model.

Some examples include replacing the machine learning model with an updated version of the machine learning model, where the updated version of the machine learning model includes the updated at least one coefficient.

In some aspects, the one or more recommended patient actions include a next best action for the patient to take in connection with closing a care gap from the plurality of identified care gaps.

In some aspects, the next best action is associated with closing more than one care gap from the plurality of identified care gaps.

In some aspects, the next best action corresponds to an action that is predicted most likely to be taken by the patient.

In some aspects, the next best action corresponds to at least one of the following: a visit to a healthcare provider, a change in a medical treatment, a change in a prescription, a change in diet, a change in activity, a blood test, and a medical examination.

Some examples include: waiting a predetermined amount of time after transmitting the electronic communication; after the predetermined amount of time, performing a patient lookback to determine whether the patient took the next best action within the predetermined amount of time; if the patient took the next best action within the predetermined amount of time, determining whether the next best action resulted in a partial or complete closing of the care gap; and updating a recommendation library based on whether the next best action results in a partial or complete closing of the care gap.

Some examples include: automatically generating a recommendation summary that includes a summarized description of the one or more recommended patient actions for the patient; and including the recommendation summary in the electronic communication.

In some aspects, the recommendation summary is generated, at least in part, with a natural language generation (NLG) model.

In some aspects, the one or more recommended patient actions for the patient are generated, at least in part, using a heterogeneous treatment effect (HTE) model.

In some aspects, the HTE model is updated using a reinforcement learning-based formulation that dynamically updates the HTE model based on clinician feedback while the HTE model is being used to generate the one or more recommended patient actions.

In some aspects, the one or more recommended patient actions includes a description of where to send the patient to address a care gap from the plurality of identified care gaps.

Some examples include: determining a social determinant of health (SDoH) for the patient; and generating at least one additional recommended patient action for the patient based on the determined SDoH for the patient, where the at least one additional recommended patient action for the patient provides a description of an action for a clinician to address a barrier for the patient using an SDoH resource, where the electronic communication describes the at least one additional recommended patient action.

All aspects, examples, and features mentioned above can be combined in any technically possible way.

### BRIEF DESCRIPTION OF THE DRAWINGS

The present disclosure is described in conjunction with the appended figures, which are not necessarily drawn to scale:

FIG. 1 illustrates an example of a system that supports providing a care plan in accordance with aspects of the present disclosure.

FIG. 2 illustrates an example diagram that supports providing a care plan in accordance with aspects of the present disclosure.

FIG. 3 illustrates an example diagram that supports providing a care plan in accordance with aspects of the present disclosure.

FIGS. 4A through 4F illustrates example diagrams and tables that support providing a care plan in accordance with aspects of the present disclosure.

FIG. 5 illustrates an example process flow that supports providing a care plan in accordance with aspects of the present disclosure.

FIG. 6 illustrates an example of a process flow that supports providing a care plan in accordance with aspects of the present disclosure.

FIGS. 7A and 7B illustrate examples that support providing a care plan in accordance with aspects of the present disclosure.

FIG. 8 illustrates an example user interface that supports providing a care plan in accordance with aspects of the present disclosure.

FIG. 9 illustrates an example process flow that supports providing a care plan in accordance with aspects of the present disclosure.

FIGS. 10A and 10B illustrate examples that support providing a care plan in accordance with aspects of the present disclosure.

FIGS. 11A through 11C illustrate an example process flow and example tables that support providing a care plan in accordance with aspects of the present disclosure.

FIG. 12 illustrates an example table of example data in accordance with aspects of the present disclosure.

FIG. 13 illustrates an example table of care opportunities in accordance with aspects of the present disclosure.

### DETAILED DESCRIPTION

Before any examples of the disclosure are explained in detail, it is to be understood that the disclosure is not limited in its application to the details of construction and the arrangement of components set forth in the following description or illustrated in the following drawings. The disclosure is capable of other configurations and of being practiced or of being carried out in various ways. Also, it is to be understood that the phraseology and terminology used herein is for the purpose of description and should not be regarded as limiting.

While various examples of generating a care plan for a member or patient will be described in connection with a member or patient, it should be appreciated that the disclosure is not so limited. For instance, it is contemplated that examples of the present disclosure can be applied to generate care plans for many different types for members or patients having any number of different conditions that could benefit from care or treatment adherence. In other words, the framework described herein for generating a care plan can be leveraged to support care management opportunities and/or manage any type or number of different medical conditions. Examples of such medical conditions that can be addressed or improved with the framework described herein include, without limitation, cardiac conditions, heightened cholesterol, heightened blood pressure, hypertension, post-operative conditions, pre-operative conditions, cancer and other chronic conditions, infertility, chronic pain, broken bones, torn ligaments, torn muscles, etc.

The terms “member”, “patient”, and “subject” may be used interchangeably herein. The terms “care gap” or “gap-in-care” may be used interchangeably herein. The terms “care manager” and “healthcare manager” may be used interchangeably herein. The terms “recommendation library” and “opportunity library” may be used interchangeably herein. The terms “care recommendation”, “recommended action”, and “recommendation” may be used interchangeably herein.

Aspects of the present disclosure support a healthcare system capable of generating healthcare recommendations that are specific, measurable, actionable, reportable, and time bound. In some healthcare systems, a care manager may be presented with an overabundance of information (e.g., information overload) to digest or analyze when viewing health histories of members. Accordingly, for example, the care manager may be unable to effectively identify a care plan for the member. For example, analyzing such an overabundance of information for each member, especially if manually implemented, may be overly time consuming.

In some cases, such analysis techniques may be inefficient and result in uncoordinated opportunities. For example, such analysis techniques may lack prioritization (e.g., with respect to health conditions, with respect to member priority, etc.) and personalization of opportunities. In some cases, some healthcare systems utilize action libraries (e.g., databases of recommended actions for treating a medical condition) that are not linked to care gaps associated with members. In some other cases, the action libraries may include too many non-standardized actions to choose from for treating medical conditions associated with a target member.

Aspects of the present disclosure support a healthcare system capable of providing a care plan for a member as well as a holistic member health summary (also referred to herein as a recommendation summary). The recommendation summary may include summarized member information that is easy to digest (e.g., by a care manager, a healthcare provider, etc.), which would basically act as the underlying context related to the generated care plan/recommendations. To this end, the healthcare system may provide artificial intelligence (AI)-based personalized and prioritized recommendations. In an example, the healthcare system may provide a consolidation of care opportunities from various sources (e.g., care considerations, next best actions) to form a comprehensive recommendation library. For example, the healthcare system may support the generation of recommendations that are personalized and prioritized per member (e.g., per member needs). In some aspects, the healthcare system may support generating a health summary and sending the health summary to providers, so that the providers can engage the members on behalf of the healthcare system. Aspects described herein support a selection process on who to send the summary to (e.g., which providers receive the health summary).

The healthcare system includes example implementations of an AI-based clinical decision support solution that integrates care opportunities from the various sources (e.g., care considerations, next best actions, etc.) to generate personalized and prioritized recommendations along with member health summaries to support care manager case preparation and care planning. In some examples, the AI-based clinical decision support solution may support advanced machine learning algorithms to generate holistic member health summaries from various data sources (e. g. tele-home assessments, in-home assessments, claims, assessments, labs, SDoH, etc.). AI-based clinical decision support solutions described herein may include causal inference modeling to generate personalized and prioritized recommendations. Examples of providing a care plan for a member according to the present disclosure are later described herein with respect to the following figures.

Aspects of providing a care plan for a member according to the present disclosure may provide improved case management compared to some healthcare systems. For example, the techniques described herein may provide reduced case preparation time by a care manager (e.g., reduced preparation time of a care plan for a member) and improved care manager efficiency. For example, a care manager may not need to contact a patient to obtain the patient’s data and thus, reducing the case preparation time for the care manager. In another example, the present disclosure may provide top recommendations and context for the top recommendations such that the care manager does not need to analyze why a recommendation was provided. Thus, care manager efficiency is increased. In some aspects, the techniques described herein may provide opportunities for the care manager to focus on the most relevant and impactful opportunities associated with addressing care gaps associated with a member. In some examples, the techniques described herein may support improved health outcomes and behavioral change for members.

For example, by synthesizing the most relevant and impactful opportunities according to a priority order, the healthcare system may support the ability of a care manager to focus on behavioral changes of members. In some examples, the healthcare system enables effective tracking of care manager actions, and the healthcare system may support determining how/which care manager care planning activities result in improved member health outcomes (e.g., improved health conditions, reduced medical costs, reduced medical visits, etc.).

Additional aspects of the present disclosure support providing Social Determinants of Health (SDoH) recommendations in association with improving clinical care gap closure, for example, in the context of generating healthcare recommendations as described herein. In an example, though some SDoH barriers may be hinder clinical care gap closure associated with a member, some healthcare systems do not provide facilities for a care manager to provide real time feedback/disposition as to the relevancy of SDoH barriers to care of the member (e.g., relevancy associated with care gap closure). For example, some healthcare systems do not provide a detailed context of which SDoH barriers affect which care gap closures.

Aspects of the present disclosure support a healthcare system capable of addressing member specific relevant SDoH barriers related to care recommendations (e.g., healthcare recommendations described herein that are specific, measurable, actionable, reportable, and time bound), which may provide improved care gap closure compared to some healthcare systems. For example, aspects of the present disclosure may provide improved support to care managers by providing relevant SDoH recommendations and resources specific to care recommendations, in association with improving care gap closure.

In some examples, the healthcare system may leverage relevant SDoH variables and indices from available member-level data and/or member-level SDoH indices (e.g., accessed from electronic records of members). For example, the healthcare system may analyze the impact of member-level SDoH barriers with respect to clinical care gaps (e.g., care recommendations described herein) to generate SDoH recommendations.

In leveraging relevant SDoH variables and indices (e.g., member-level SDoH barriers), aspects of the healthcare system described herein include utilizing AI-based clinical decision support solutions described herein with reference to generating healthcare recommendations (e.g., healthcare recommendations that are specific, measurable, actionable, reportable, and time bound). In an example, the healthcare system may utilize AI-based causal inference modeling. In some aspects, the healthcare system may connect (e.g., correlate) SDoH recommendations with the SDoH resource library information, which may provide features that support the ability of a care manager to address care gaps resulting due to SDoH variables.

For example, a care recommendation for a member may include a recommended visit to a primary care provider. Aspects of the present disclosure associated with addressing SDoH gaps may support identifying that the member may need transportation support. For example, the healthcare system may identify that a transportation barrier was found to be impacting the primary care provider visit for the member, and that a community shuttle service may help in closing the gap in care. In another example, a care recommendation for a member may include increased exercise and the healthcare system may identify one or more exercise programs to enroll the member in (e.g., group exercise classes, exercise videos, etc.).

Examples of providing SDoH recommendations in association with improving clinical care gap closure according to the present disclosure are later described herein with respect to the following figures.

Aspects of providing SDoH recommendations in association with improving clinical care gap closure according to the present disclosure may provide improved healthcare outcomes (e.g., improved care gap closure, improved behavioral changes, etc.) for members, compared to some healthcare systems. For example, the techniques described herein may support increased member engagement through providing personalized SDoH resources.

Example aspects of the present disclosure are described with reference to the following figures.

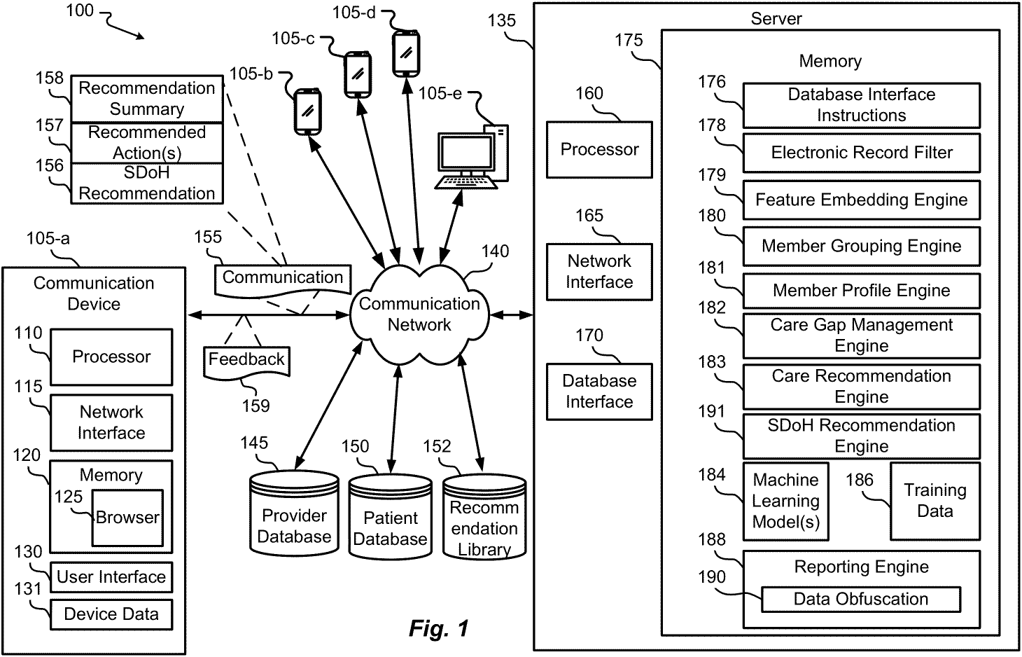

FIG. 1 illustrates an example of a system 100 that supports providing a care plan in accordance with aspects of the present disclosure. The system 100, in some examples, may include one or more computing devices operating in cooperation with one another to determine recommended actions for a member based on, for example, treatment effect scores corresponding to identified care gaps. The system 100 may be, for example, a healthcare management system.

The components of the system 100 may be utilized to facilitate one, some, or all of the methods described herein or portions thereof without departing from the scope of the present disclosure. Furthermore, the servers described herein may include example components or instruction sets, and aspects of the present disclosure are not limited thereto. In an example, a server may be provided with all of the instruction sets and data depicted and described in the server of FIG. 1. Alternatively, or additionally, different servers or multiple servers may be provided with different instruction sets than those depicted in FIG. 1.

The system 100 may include communication devices 105 (e.g., communication device 105-a through communication device 105-e), a server 135, a communication network 140, a provider database 145, a member database 150, and a recommendation library 152. The communication network 140 may facilitate machine-to-machine communications between any of the communication device 105 (or multiple communication devices 105), the server 135, or one or more databases (e.g., a provider database 145, a member database 150, a recommendation library 152). The communication network 140 may include any type of known communication medium or collection of communication media and may use any type of protocols to transport messages between endpoints. The communication network 140 may include wired communications technologies, wireless communications technologies, or any combination thereof.

The Internet is an example of the communication network 140 that constitutes an Internet Protocol (IP) network consisting of multiple computers, computing networks, and other communication devices located in multiple locations, and components in the communication network 140 (e.g., computers, computing networks, communication devices) may be connected through one or more telephone systems and other means. Other examples of the communication network 140 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 140 may include of any combination of networks or network types. In some aspects, the communication network 140 may include any combination of communication mediums such as coaxial cable, copper cable/wire, fiber-optic cable, or antennas for communicating data (e.g., transmitting/receiving data).

A communication device 105 (e.g., communication device 105-a) may include a processor 110, a network interface 115, a computer memory 120, a user interface 130, and device data 131. In some examples, components of the communication device 105 (e.g., processor 110, network interface 115, computer memory 120, user interface 130) may communicate over a system bus (e.g., control busses, address busses, data busses) included in the communication device 105. In some cases, the communication device 105 may be referred to as a computing resource. The communication device 105 may establish one or more connections with the communication network 140 via the network interface 115. In some cases, the communication device 105 may transmit or receive packets to one or more other devices (e.g., another communication device 105, the server 135, the provider database 145, the provider database 150, the recommendation library 152, etc.) via the communication network 140.

Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user.

The communication device 105 may support one or more operations or procedures associated with providing a care plan for a member. For example, the communication device 105 may support communications between multiple entities such as a healthcare provider (e.g., a nurse, a care manager, a physician, etc.), a patient (also referred to herein as a member), or combinations thereof. In some cases, the system 100 may include any number of communication devices 105, and each of the communication devices 105 may be associated with a respective entity.

The communication device 105 may render or output any combination of notifications, messages, reports, menus, etc. based on data communications transmitted or received by the communication device 105 over the communication network 140. For example, the communication device 105 may receive one or more electronic communications 155 (e.g., from the server 135) via the communication network 140.

In some aspects, the communication device 105 may render a presentation (e.g., visually, audibly, using haptic feedback, etc.) of the electronic communication 155 via the user interface 130. The user interface 130 may include, for example, a display, an audio output device (e.g., a speaker, a headphone connector), or any combination thereof. In some aspects, the communication device 105 may render a presentation using one or more applications (e.g., a browser application 125) stored on the memory 120. In an example, the browser application 125 may be configured to receive the electronic communication 155 in an electronic format (e.g., in an electronic communication via the communication network 140) and present content of the electronic communication 155 via the user interface 130.

In some aspects, the electronic communication 155 may be a communication including a recommended action(s) 157 for a member that, if followed, is capable of at least partially closing a current gap-in-care within a clinically-defined period of time for the member. In some aspects, the electronic communication 155 may include a recommendation summary 158. In some aspects, the electronic communication 155 may include an SDoH recommendation 156. Examples of the SDoH recommendation 156, the recommended action(s) 157 and the recommendation summary 158 are later described herein.

The recommendation library 152 may include candidate actions (e.g., candidate care recommendations) from which the recommended action(s) 157 may be selected. Example aspects of the recommendation library 152 are later described herein.

In some aspects, the server 135 may communicate the electronic communication 155 to a communication device 105 (e.g., communication device 105-a) of a member, a communication device 105 (e.g., communication device 105-b) of a healthcare provider, a communication device 105 (e.g., communication device 105-c) of an insurance provider, a communication device 105 (e.g., communication device 105-d) of a pharmacist or pharmacy, a communication device 105 (e.g., communication device 105-e) of team outreach personnel, or the like. Additionally, or alternatively, the server 135 may communicate a physical representation (e.g., a letter) of the electronic communication 155 to the member, a healthcare provider, an insurance provider, a pharmacist, team outreach personnel, or the like via a direct mail provider (e.g., postal service). In some aspects, the SDoH recommendation 156, the recommended action(s) 157, and the recommendation summary 158 may be accessible (e.g., by a member, a healthcare provider, etc.) via an application (e.g., a mobile application, a native application, browser 125, etc.) executed at a communication device 105.

The provider database 145 and the member database 150 may include member electronic records (also referred to herein as a data records) stored therein. In some aspects, the electronic records may be accessible to a communication device 105 (e.g., operated by healthcare provider personnel, insurance provider personnel, a member, a pharmacist, etc.) and/or the server 135. In some aspects, a communication device 105 and/or the server 135 may receive and/or access the electronic records from the provider database 145 and the member database 150 (e.g., based on a set of permissions).

In some aspects, an electronic record associated with a member may include claims-based electronic data. For example, the electronic record may include electronic medical record (EMR) data. In another example, the claims-based electronic data may include data describing an insurance medical claim, pharmacy claim, and/or insurance claim made by the member and/or a medical provider. Accordingly, for example, the claims-based electronic data may come from providers or payers, and claims included in the claims-based electronic data may be of various types (e.g., medical, pharmacy, etc.).

In some other aspects, the electronic record associated with the member may include device data 131 obtained from a communication device 105 (e.g., communication device 105-a) associated with the member. For example, the device data 131 may include gyroscopic data, accelerometer data, beacon data, glucose readings, heart rate data, blood pressure data, blood oxygen data, temperature data, kinetics data, location data, motion data, a device identifier, and/or temporal data (e.g., a timestamp) measurable, trackable, and/or providable by the communication device 105 (or a device connected to the communication device 105) associated with the member.

In some aspects, the electronic record may include an image of the member. For example, the electronic record may include imaging data based on which the server 135 (e.g., the care gap management engine 182) may track targeted biomarkers. For example, the server 135 may track X-ray records of a member over time (e.g., in associated with assisting reduced healing times for a member). In some cases, the electronic record may include other types of diagnostic images such as magnetic resonance imaging (MRI) scans, computed tomography scans (CT), ultrasound images, or the like.

In accordance with aspects of the present disclosure, the device data 131 may be provided continuously, semi-continuously, periodically, and/or based on a trigger condition by the communication device 105 (e.g., a smart watch, a wearable monitor, a self-reporting monitor such as a glucometer, a smartphone carried by a user, etc.) around monitored parameters such as heartbeat, blood pressure, etc. In some aspects, the device data 131 of a communication device 105 (e.g., communication device 105-a) may be referred to as “environmental data” associated with a user, which may be representative of aspects of environmental factors (e.g., lifestyle, socioeconomic factors, details about the environment, etc.) associated with a member.

In some cases, the device data 131 may include wearable-device data, glucose readings, heart rate, body temperature, “invisible” data (e.g., device related information associated with a member, such as Bluetooth beacon information), and/or self-reporting monitored data (e.g., provided by self-reporting biometric meters).

In some aspects, the electronic record may include genetic data associated with a member. In some other aspects, the electronic record may include notes/documentation that is recorded at a communication device 105 in a universal and/or systematic format (e.g., subjective, objective, assessment, and plan (SOAP) notes/documentation) among medical providers, insurers, etc. In some examples, the electronic record may include non-claim adjudicated diagnoses input at a communication device 105 (e.g., diagnoses that have not been evaluated by an insurance provider with respect to payment of benefits).

In some other aspects, the electronic records may be inclusive of aspects of a member’s health history and health outlook. The electronic records may include a number of fields for storing different types of information to describe the member’s health history and health outlook. As an example, the electronic records may include personal health information (PHI) data. The PHI data may be stored encrypted and may include member identifier information such as, for example, name, address, member number, social security number, date of birth, etc. In some aspects, the electronic records may include treatment data such as, for example, member health history, member treatment history, lab test results (e.g., text-based, image-based, or both), pharmaceutical treatments and therapeutic treatments (e.g., indicated using predefined healthcare codes, treatment codes, or both), insurance claims history, healthcare provider information (e.g., doctors, therapists, etc. involved in providing healthcare services to the member), in-member information (e.g., whether treatment is associated with care), location information (e.g., associated with treatments or prescriptions provided to the member), family history (e.g., inclusive of medical data records associated with family members of the member, data links to the records, etc.), or any combination thereof. In some aspects, the electronic records may be stored or accessed according to one or more common field values (e.g., common parameters such as common healthcare provider, common location, common claims history, etc.). In some aspects, the system 100 may support member identifiers based on which a server 135 and/or a communication device 105 may access and/or identify key health data per member different from the PHI data.

In some aspects, the server 135 may receive the guideline behavior for the member supported by a professional clinical recommendation. For example, the server 135 may receive and/or access the guideline behavior from a communication device 105, the provider database 145, the member database 150, and/or another server 135. In some examples, the guideline behavior for the member supported by the professional clinical recommendation may include guidance based on at least one of medical history, demographics, social indices, biomarkers, behavior data, engagement data, historical gap-in-care data, and a machine learning model-derived output (e.g., a risk-based model probability derived by a machine learning model(s) 184 described herein). In some aspects, the guidance may be based on medical history, demographics, social indices, biomarkers, behavior data, engagement data, historical gap-in-care data, and/or machine learning model-derived output(s) that correspond to the member and/or other members.

In some aspects, the gap-in-care described herein may be defined by a difference between guideline behavior associated with what a member should be doing, as defined by clinical guidelines and expert clinical opinion (e.g., professional guidelines surrounding preventative screenings and close follow-up and monitoring with healthcare providers) and current health related behavior associated with what the member is actually doing, which may be defined by static or longitudinal observables in the medical history of the member and supporting data.

In some aspects, the provider database 145 may be accessible to a healthcare provider of a member, and in some cases, include member information associated with the healthcare provider that provided a treatment to the member. In some aspects, the provider database 145 may be accessible to an insurance provider associated with the member. The member database 150 may correspond to any type of known database, and the fields of the electronic records may be formatted according to the type of database used to implement the member database 150. Non-limiting examples of the types of database architectures that may be used for the provider database 145, the member database 150, and the recommendation library 152 include a relational database, a centralized database, a distributed database, an operational database, a hierarchical database, a network database, an object-oriented database, a graph database, a NoSQL (non-relational) database, etc. In some cases, the member database 150 may include an entire healthcare history or journey of a member, whereas the provider database 145 may provide a snapshot of a member’s healthcare history with respect to a healthcare provider. In some examples, the electronic records stored in the member database 150 may correspond to a collection or aggregation of electronic records from any combination of provider databases 145 and entities involved in the member’s healthcare delivery (e.g., a care manager, a pharmaceutical distributor, a pharmaceutical manufacturer, etc.).

The server 135 may include a processor 160, a network interface 165, a database interface 170, and a memory 175. In some examples, components of the server 135 (e.g., processor 160, a network interface 165, a database interface 170, and a memory 175) may communicate via a system bus (e.g., any combination of control busses, address busses, and data busses) included in the server 135. Aspects of the processor 160, network interface 165, database interface 170, and memory 175 may support example functions of the server 135 as described herein. For example, the server 135 may transmit packets to (or receive packets from) one or more other devices (e.g., one or more communication devices 105, another server 135, the provider database 145, the provider database 150) via the communication network 140. In some aspects, via the network interface 165, the server 135 may transmit database queries to one or more databases (e.g., provider database 145, member database 150) of the system 100, receive responses associated with the database queries, or access data associated with the database queries.

In some aspects, via the network interface 165, the server 135 may transmit one or more electronic communications 155 described herein to one or more communication devices 105 of the system 100. The network interface 165 may include, for example, any combination of network interface cards (NICs), network ports, associated drivers, or the like. Communications between components (e.g., processor 160, network interface 165, database interface 170, and memory 175) of the server 135 and other devices (e.g., one or more communication devices 105, the provider database 145, the provider database 150, the recommendation library 152, another server 135) connected to the communication network 140 may, for example, flow through the network interface 165.

The processors described herein (e.g., processor 110 of the communication device 105, processor 160 of the server 135) may correspond to one or many computer processing devices. For example, the processors may include a silicon chip, such as a Field Programmable Gate Array (FPGA), an Application-Specific Integrated Circuit (ASIC), any other type of Integrated Circuit (IC) chip, a collection of IC chips, or the like. In some aspects, the processors may include a microprocessor, a Central Processing Unit (CPU), a Graphics Processing Unit (GPU), or plurality of microprocessors configured to execute the instructions sets stored in a corresponding memory (e.g., memory 120 of the communication device 105, memory 175 of the server 135). For example, upon executing the instruction sets stored in memory 120, the processor 110 may enable or perform one or more functions of the communication device 105. In another example, upon executing the instruction sets stored in memory 175, the processor 160 may enable or perform one or more functions of the server 135.

The processors described herein (e.g., processor 110 of the communication device 105, processor 160 of the server 135) may utilize data stored in a corresponding memory (e.g., memory 120 of the communication device 105, memory 175 of the server 135) as a neural network. The neural network may include a machine learning architecture. In some aspects, the neural network may be or include one or more classifiers. In some other aspects, the neural network may be or include any machine learning network such as, for example, a deep learning network, a convolutional neural network, or the like. Some elements stored in memory 120 may be described as or referred to as instructions or instruction sets, and some functions of the communication device 105 may be implemented using machine learning techniques. In another example, some elements stored in memory 175 may be described as or referred to as instructions or instruction sets, and some functions of the server 135 may be implemented using machine learning techniques.

In some aspects, the processors (e.g., processor 110, processor 160) may support machine learning model(s) 184 which may be trained and/or updated based on data (e.g., training data 186) provided or accessed by any of the communication device 105, the server 135, the provider database 145, and the member database. The machine learning model(s) 184 may be built and updated by any of the engines (e.g., member profile engine 181, care gap management engine 182, care recommendation engine 183, SDoH recommendation engine 191, etc.) described herein based on the training data 186 (also referred to herein as training data and feedback). For example, the machine learning model(s) 184 may be trained with feature vectors of members (e.g., accessed from provider database 145 or member database 150) for which adherence to one or more recommended actions 157 (e.g., care recommendations) reduced a corresponding gap-in-care and/or achieved one or more impacts (e.g., cost impact, clinical impact, reduction in inpatient visits, etc.). In some examples, the machine learning model(s) 184 may be trained with additional feature vectors of members (e.g., accessed from provider database 145 or member database 150) for which adherence to one or more SDoH recommendations 156 reduced a corresponding gap-in-care and/or achieved one or more impacts.

In some aspects, the training data 186 may include multiple training sets. For example, the machine learning model(s) 184 may be trained with a first training set that includes feature vectors of members (e.g., accessed from provider database 145 or member database 150) for which adherence to one or more recommended action(s) 157 (e.g., tracking asthma peak flow, BMI assessment, HBA1C monitoring, tracking blood pressure) achieved one or more relatively positive impacts (e.g., positive cost impact, positive clinical impact, reduction in number of inpatient visits over a predetermined period, etc.). In an example, the first training set may include feature vectors of members for which adherence to one or more actions reduced a corresponding gap-in-care.

In an example, the machine learning model(s) 184 may be trained with a second training set that includes feature vectors of members for which a failure to adhere to one or more recommended action(s) 157 resulted in a relatively negative impact (e.g., negative cost impact, negative clinical impact, no reduction in number of inpatient visits over a predetermined period, an increase in number of inpatient visits over the predetermined period, etc.).

In some other examples, aspects of the present disclosure include creating a third training set based on data included in any of the first and second training sets. For example, generating the third training set may include identifying treatment effects, statistical significances, opportunity values, and ranking information associated with the one or more recommended action(s) 157.

In some examples, aspects of the present disclosure include training the machine learning model(s) 184 with a fourth training set that includes feature vectors of members that did not have any SDoH barriers preventing the members from adhering to one or more recommended action(s) 157, and for which adhering to the one or more recommended action(s) 157 achieved one or more relatively positive impacts (e.g., positive cost impact, positive clinical impact, care gap closure within a predetermined period of time, etc.).

In another example, aspects of the present disclosure include training the machine learning model(s) 184 with a fifth training set that includes feature vectors of members that had an SDoH barrier(s) preventing the members from adhering to one or more recommended action(s) 157, and for which a failure to adhere to the one or more recommended action(s) 157 (e.g., due to the SDoH barrier(s)) resulted in a relatively negative impact (e.g., negative cost impact, negative clinical impact, no care gap closure within a predetermined period of time, etc.).

In some other examples, aspects of the present disclosure include creating a sixth training set based on data included in any of the fourth and fifth training sets. For example, generating the sixth training set may include identifying treatment effects and/or statistical significances associated with a recommended action(s) 157 and an SDoH barrier(s).

In another example, aspects of the present disclosure include training the machine learning model(s) 184 with a seventh training set that includes feedback 159 (e.g., clinical feedback, care manager feedback, and assessment data, etc.) described herein. Examples of feedback 159 are later described herein.

Aspects of the present disclosure include training the machine learning model(s) 184 with an additional training set(s) that includes feature vectors of members for which adherence to one or more recommended action(s) 157 still failed to achieve a target impact. For example, the additional training set(s) may include additional factors (e.g., outside of adherence to the one or more actions) that correlate to the failure to achieve the target impact.

In some other examples, aspects of the present disclosure include creating additional training sets based on data included in any of the training sets described herein. For example, generating the additional training sets may include identifying a relatively larger set of factors (e.g., member characteristics, environmental factors, actions, etc.) that may affect whether a target impact is achievable.

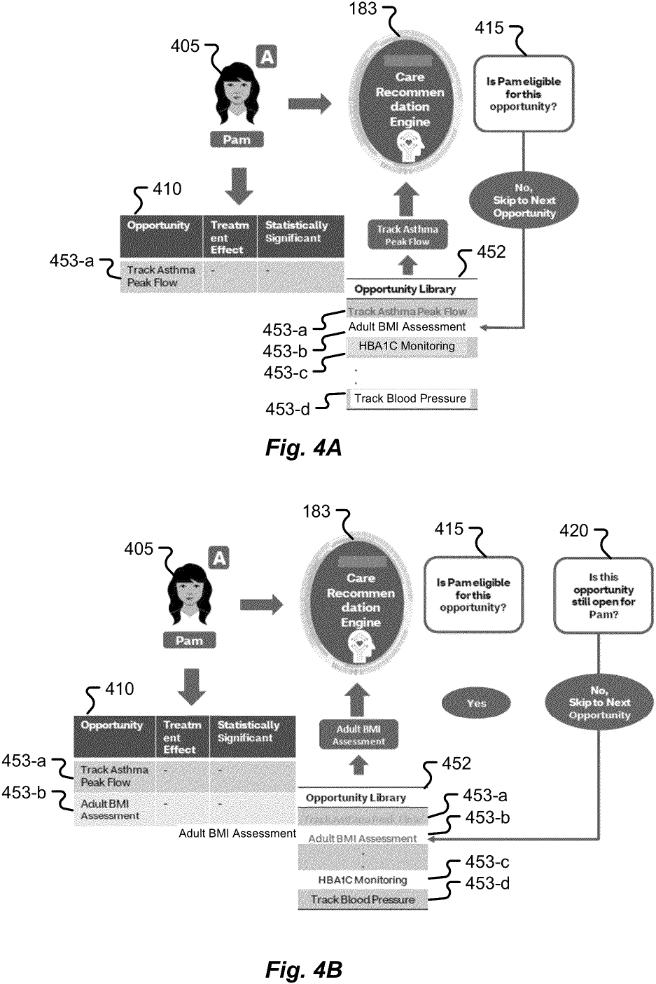

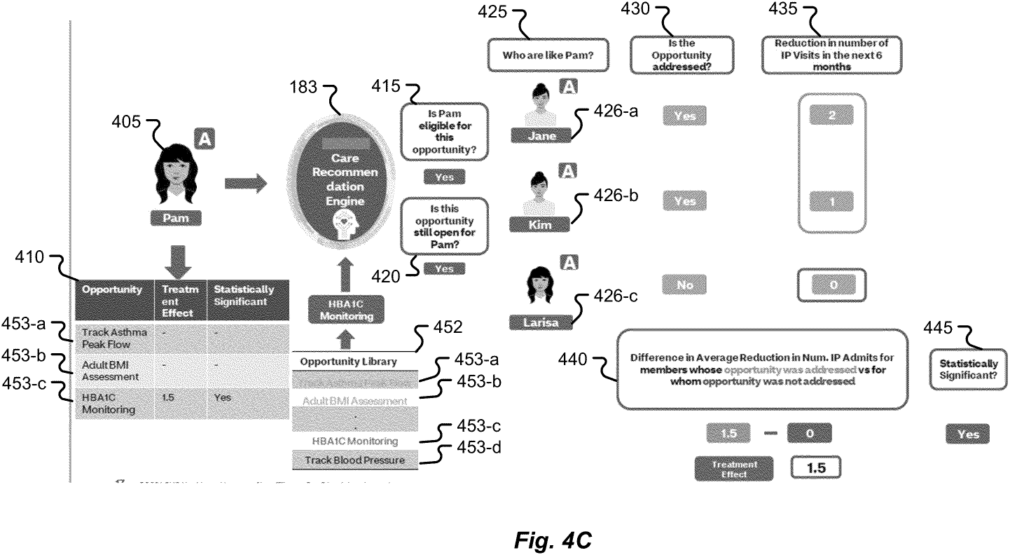

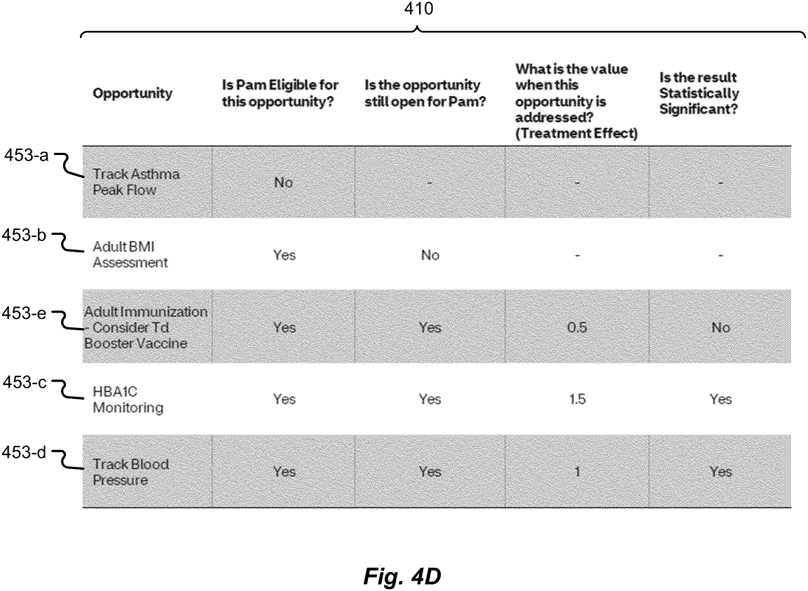

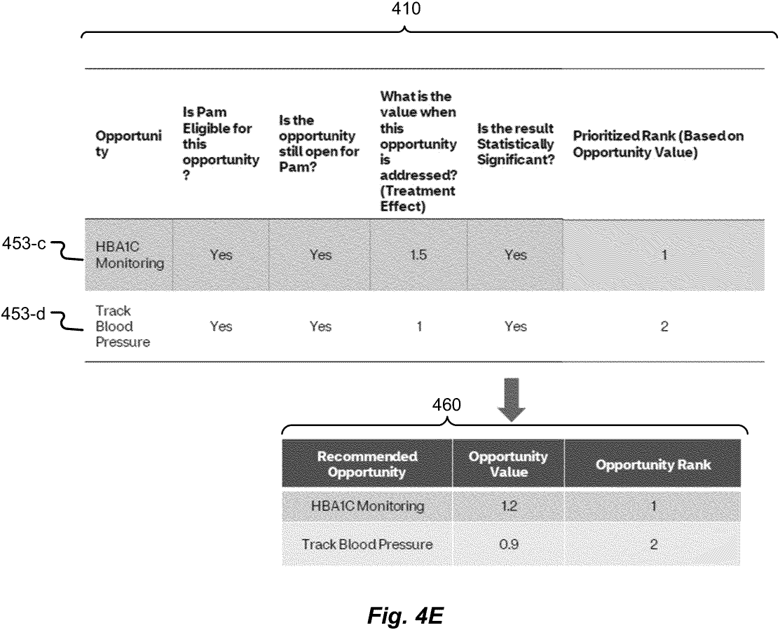

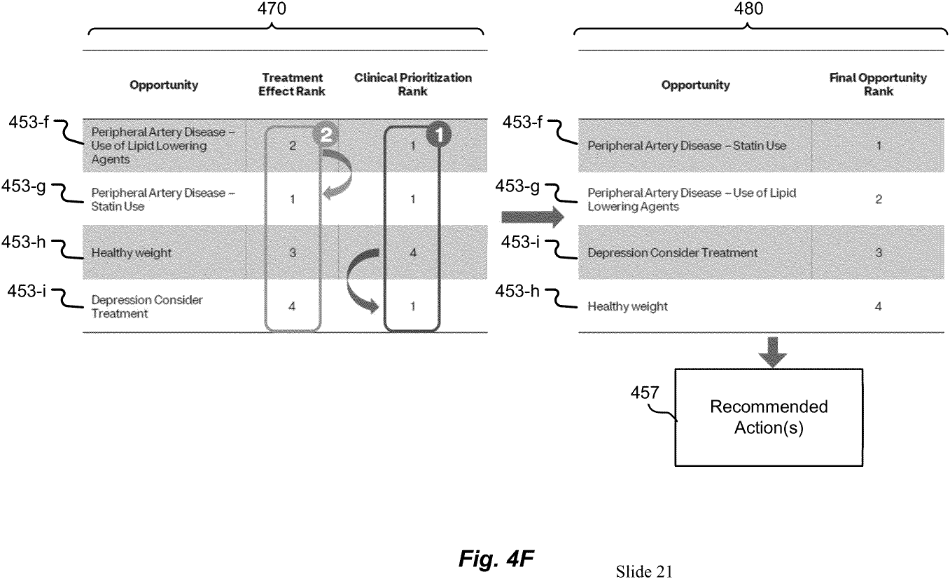

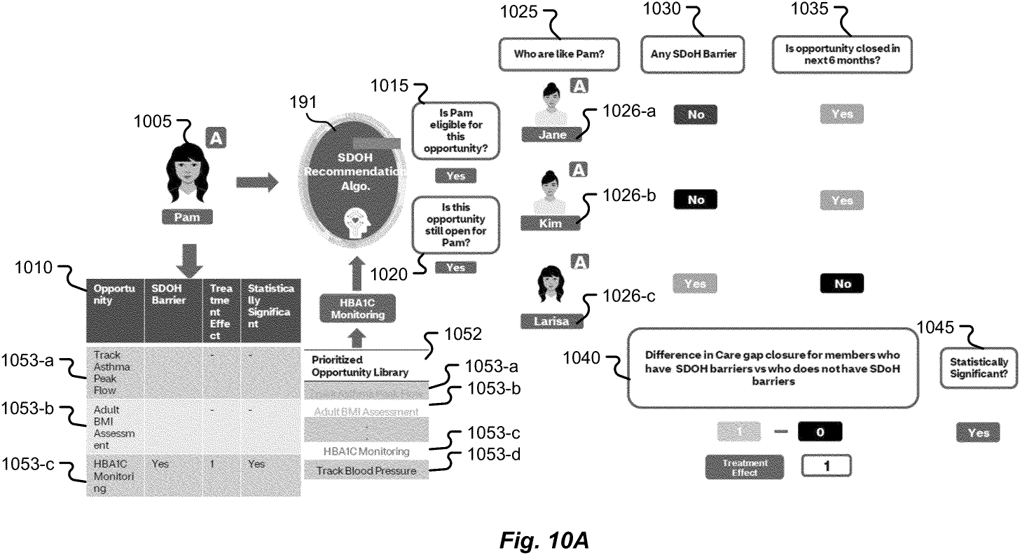

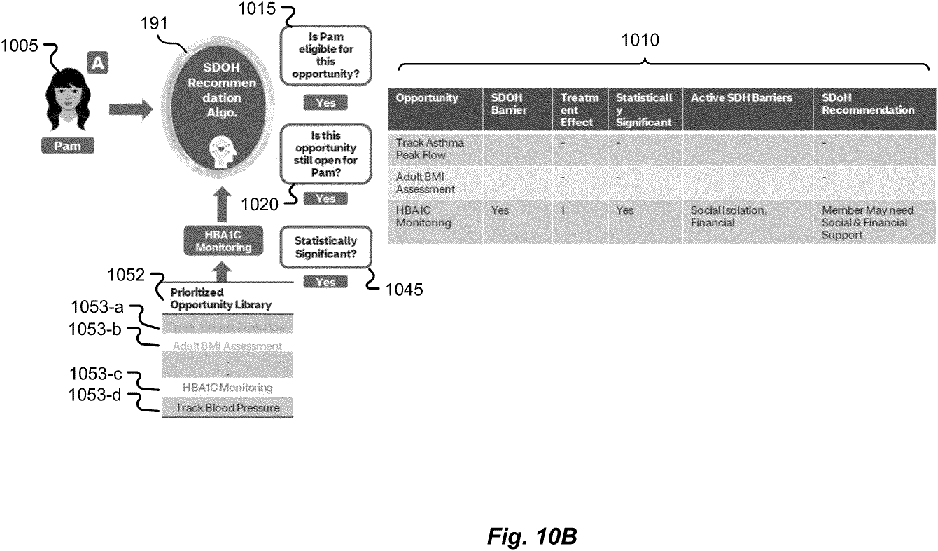

Example aspects of the training sets are later described, for example, with reference to FIGS. 4A through 4F and FIGS. 10A and 10B.

The machine learning model(s) 184 may be provided in any number of formats or forms. Example aspects of the machine learning model(s) 184, such as generating (e.g., building, training) and applying the machine learning model(s) 184, are described with reference to the figure descriptions herein.

Non-limiting examples of the machine learning model(s) 184 include Decision Trees, gradient-boosted decision tree approaches (GBMs), Support Vector Machines (SVMs), Nearest Neighbor, and/or Bayesian classifiers, and neural-network-based approaches. Another example of the machine learning model(s) 184 includes a natural language generation (NLG) model. Another example of the machine learning model(s) 184 includes a heterogeneous treatment effect (HTE) model. In some aspects, the HTE model may be updated using a reinforcement learning-based formulation that dynamically updates the HTE model based on clinician feedback (e.g., feedback 159) while the HTE model is being used to generate recommended patient actions (e.g., recommended action(s) 157).

In some examples, based on the data (e.g., training data 186), the neural network may generate one or more algorithms described herein supportive of generating a patient care plan described herein.

In some aspects, the machine learning model(s) 184 may include ensemble classification models (also referred to herein as ensemble methods) such as gradient boosting machines (GBMs). Gradient boosting techniques may include, for example, the generation of decision trees one at a time within a model, where each new tree may support the correction of errors generated by a previously trained decision tree (e.g., forward learning). Gradient boosting techniques may support, for example, the construction of ranking models for information retrieval systems. A GBM may include decision tree-based ensemble algorithms that support building and optimizing models in a stage-wise manner.

According to example aspects of the present disclosure described herein, the machine learning model(s) 184 may include Gradient Boosting Decision Trees (GBDTs). Gradient boosting is a supervised learning technique that harnesses additive training and tree boosting to correct errors made by previous models, or regression trees.

The machine learning model(s) 184 may include extreme gradient boosting (CatBoost) models. CatBoost is an ensemble learning method based on GBDTs. In some cases, CatBoost methods may have improved performance compared to comparable random forest-based methods. CatBoost methods are easily tunable and scalable, offer a higher computational speed in comparison to other methods, and are designed to be highly integrable with other approaches including Shapley Additive Explanations (SHAP) values

In some aspects, the machine learning model(s) 184 may include ensemble classification models (also referred to herein as ensemble methods) such as random forests. Random forest techniques may include independent training of each decision tree within a model, using a random sample of data. Random forest techniques may support, for example, medical diagnosis techniques described herein using weighting techniques with respect to different data sources. Various example aspects of the machine learning model(s) 184, inputs to the machine learning model(s) 184, and the training data 186 with respect to the present disclosure are described herein.

The memory described herein (e.g., memory 120, memory 175) may include any type of computer memory device or collection of computer memory devices. For example, a memory (e.g., memory 120, memory 175) may include a Random Access Memory (RAM), a Read Only a Memory (ROM), a flash memory, an Electronically-Erasable Programmable ROM (EEPROM), Dynamic RAM (DRAM), or any combination thereof.

The memory described herein (e.g., memory 120, memory 175) may be configured to store instruction sets, neural networks, and other data structures (e.g., depicted herein) in addition to temporarily storing data for a respective processor (e.g., processor 110, processor 160) to execute various types of routines or functions. For example, the memory 175 may be configured to store program instructions (instruction sets) that are executable by the processor 160 and provide functionality of any of the engines (e.g., feature embedding engine 179, member grouping engine 180, member profile engine 181, care gap management engine 182, care recommendation engine 183, SDoH recommendation engine 191, reporting engine 188, etc.) described herein.

The memory described herein (e.g., memory 120, memory 175) may also be configured to store data or information that is useable or capable of being called by the instructions stored in memory. Examples of data that may be stored in memory 175 for use by components thereof include machine learning model(s) 184 and/or training data 186 described herein.

Any of the engines described herein may include a single or multiple engines.

With reference to the server 135, the memory 175 may be configured to store instruction sets, neural networks, and other data structures (e.g., depicted herein) in addition to temporarily storing data for the processor 160 to execute various types of routines or functions. The illustrative data or instruction sets that may be stored in memory 175 may include, for example, database interface instructions 176, an electronic record filter 178 (also referred to herein as a feature vector filter), a feature embedding engine 179, a care gap management engine 182, and a reporting engine 188. In some examples, the reporting engine 188 may include data obfuscation capabilities 190 via which the reporting engine 188 may obfuscate, remove, redact, or otherwise hide personally identifiable information (PII) from an electronic communication 155 prior to transmitting the electronic communication 155 to another device (e.g., communication device 105).

In some examples, the database interface instructions 176, when executed by the processor 160, may enable the server 135 to send data to and receive data from the provider database 145, the member database 150, or both. For example, the database interface instructions 176, when executed by the processor 160, may enable the server 135 to generate database queries, provide one or more interfaces for system administrators to define database queries, transmit database queries to one or more databases (e.g., provider database 145, the member database 150), receive responses to database queries, access data associated with the database queries, and format responses received from the databases for processing by other components of the server 135.

The server 135 may use the electronic record filter 178 in connection with processing data received from the various databases (e.g., provider database 145, member database 150). For example, the electronic record filter 178 may be leveraged by the database interface instructions 176 to filter or reduce the number of electronic records (e.g., feature vectors) provided to any of the engines described herein. In an example, the database interface instructions 176 may receive a response to a database query that includes a set of feature vectors (e.g., a plurality of feature vectors associated with different members). In some aspects, any of the database interface instructions 176 or the engines described herein may be configured to utilize the electronic record filter 178 to reduce (or filter) the number of feature vectors received in response to the database query, for example, prior to processing data included in the feature vectors.

The feature embedding engine 179 may receive, as input, sequences of medical terms extracted from claim data (e.g., medical claims, pharmacy claims) for each member. In an example, the feature embedding engine 179 may process the input using neural word embedding algorithms such as Word2vec. In some examples, the feature embedding engine 179 may process the input using Transformer algorithms (e.g., algorithms associated with language models such as Bidirectional Encoder Representations from Transformers (BERT) or Generative Pre-trained Transformer (GPT) or graph convolutional transformer (GCT)) and respective attentional mechanisms. In some aspects, based on the processing, the feature embedding engine 179 may compute and output respective dimension weights for the medical terms. In some aspects, the dimension weights may include indications of the magnitude and direction of the association between a medical code and a dimension. In an example, the feature embedding engine 179 may compute an algebraic average of all the medical terms for each member over any combination of dimensions (e.g., over all dimensions). In some aspects, the algebraic average may be provided by the feature embedding engine 179 as additional feature vectors in a predictive model described herein (e.g., classifier).

The member grouping engine 180, when executed by the processor 160, may enable the server 135 to group data records of various members according to a common value(s) in one or more fields of such data records. For example, the member grouping engine 180 may group electronic records based on commonalities in parameters such as health conditions (e.g., diagnosis of diabetes, open gaps-in-care, closed gaps-in-care, suggested actions associated with closing a gap-in-care, impact associated with at least partially closing the gap-in-care, etc.), medical treatment histories, prescriptions, healthcare providers, locations (e.g., state, city, ZIP code, etc.), gender, age range, medical claims, pharmacy claims, lab results, medication adherence, demographic data, social determinants (also referred to herein as social indices or SDoH), biomarkers, behavior data, engagement data, historical gap-in-care data, machine learning model-derived outputs, combinations thereof, and the like.

The member profile engine 181 may process (e.g., using a machine learning model(s) 184) data inputs including electronic patient data obtained from a plurality of electronic records describing a health history of a member. In an example, the member profile engine 181 may generate member profiles and/or profile summaries associated with the member. Example aspects of the member profile engine 181 are later described herein.

The care gap management engine 182 may process (e.g., using a machine learning model(s) 184) data inputs including electronic patient data obtained from a plurality of electronic records describing a health history of a member(s). In an example, the care gap management engine 182 may generate a plurality of identified care gaps for the member(s). Example aspects of the care gap management engine 182 are later described herein.

The care recommendation engine 183 may generate care recommendations (e.g., recommended action(s) 157, recommendation summary 158, etc.) for the member. In an example, the care recommendations may be specific, measurable, actionable, reportable, and time bound. Example aspects of the care recommendation engine 183 are later described herein.

The SDoH recommendation engine 191 may provide an SDoH recommendation(s) 156 linked to recommended action(s) 157 provided by the care recommendation engine 183. In an example, the SDoH recommendation(s) 156 may indicate, for a recommended action(s) 157, whether additional assistance (e.g., social support, transportation support, financial support, etc.) should be provided to the member in order for the member to adhere to the recommended action(s) 157. For example, the SDoH recommendation engine 191 may identify that a transportation barrier was found to be impacting the primary care provider visit for the member, and that a community shuttle service may help in closing the gap in care. In another example, the care recommendation for a member may include increased exercise and the SDoH recommendation engine 191 may identify one or more exercise programs to enroll the member in (e.g., group exercise classes, exercise videos, etc.). The enrollment may be performed automatically (e.g., without user input) by the SDoH recommendation engine 191 or may be performed with manual intervention after providing the recommendation and option to the member.

Accordingly, for example, the SDoH recommendation(s) 156 may support improved adherence to the recommended action(s) 157.

The reporting engine 188, when executed by the processor 160, may enable the server 135 to output one or more electronic communications 155 (and/or provide electronic records described herein) based on data generated by any of engines described herein. The reporting engine 188 may be configured to generate electronic communications 155 (and/or provide electronic records described herein) in various electronic formats, printed formats, or combinations thereof. Some example formats of the electronic communications 155 may include HyperText Markup Language (HTML), electronic messages (e.g., email), documents for attachment to an electronic message, text messages (e.g., SMS, instant messaging, etc.), combinations thereof, or any other known electronic file format. Some other examples include sending, for example, via direct mail, a physical representation (e.g., a letter) of the electronic communication 155.

The reporting engine 188 may also be configured to hide, obfuscate, redact, or remove PII data from an electronic communication 155 prior to transmitting the electronic communication 155 to another device (e.g., a communication device 105). The reporting engine 188 may also be configured to hide, obfuscate, redact, or remove PII data from an electronic record prior to transmitting the electronic record via an electronic communication 155 to another device (e.g., a communication device 105). In some aspects, a communication device 105 may also be configured to hide, obfuscate, redact, or remove PII data from direct mail (e.g., a letter) prior to generating a physical representation (e.g., a printout) of an electronic communication 155 (and/or electronic record). In some examples, the data obfuscation may include aggregating electronic records to form aggregated member data that does not include any PII for a particular member or group of members. In some aspects, the aggregated member data generated by the data obfuscation may include summaries of data records for member groups, statistics for member groups, or the like.

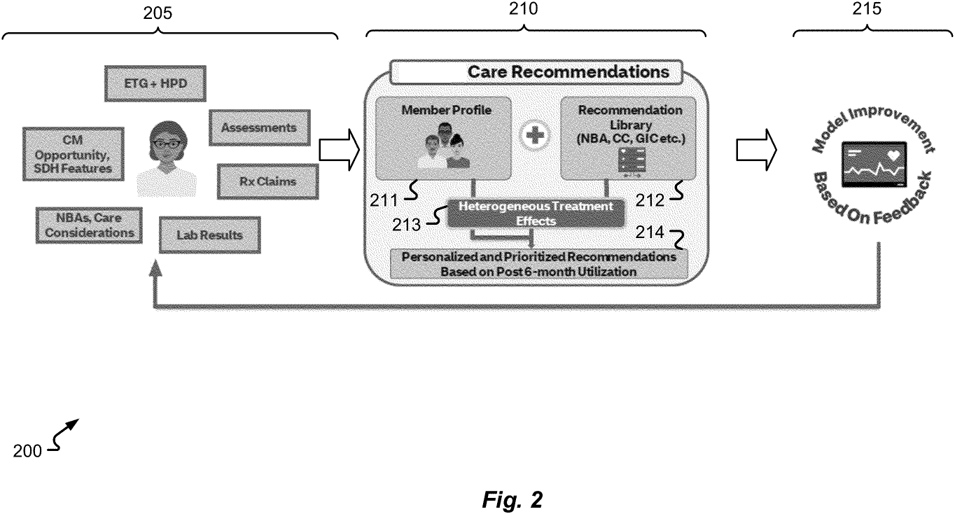

Example illustrative aspects of the system 100 are described with reference to FIGS. 2 through 13.

FIG. 2 illustrates an example 200 supportive of providing a care plan in accordance with aspects of the present disclosure. The example 200 may be implemented by aspects of the system 100 (e.g., member profile engine 181, care recommendation engine 183, etc.) described herein.

The system 100 (e.g., member profile engine 181) may create a member profile 211 from data 205 provided by various data sources. The data 205 may include, for example, Episodic Treatment Groups (ETG) + Health Profile Database (HPD), assessment data (e.g., assessments by a healthcare provider, assessments by a care manager, self-assessments by a member, etc.), pharmacy data (e.g., prescription claims data), lab results, next best actions, care considerations, care manager opportunity, and SDoH features.

The system 100 (e.g., care recommendation engine 183) may generate care recommendations 210 for the member. In an example, the care recommendations 210 may be specific, measurable, actionable, reportable, and time bound, examples of which are later described herein. Care recommendations 210 may be an example of recommended action(s) 157 described with reference to FIG. 1.

The care recommendations 210 may include personalized and prioritized recommendations 214. The personalized and prioritized recommendations 214 may be provided and recommended (e.g., by the care recommendation engine 183) from a recommendation library 212. Recommendation library 212 may include a set of candidate care recommendations from which the care recommendations 210 may be selected by the system 100. Recommendation library 212 may include aspects of recommendation library 152 described with reference to FIG. 1.

In an example, the care recommendation engine 183 may identify heterogeneous treatment effects 213 based on data included in the member profile 211 and the recommendation library 212. Heterogeneous treatment effects 213 may include treatment effects that are generated by using a doubly robust causal inference model. The doubly robust causal inference model may compute the treatment effect score for closing each care gap (i.e., each recommendation in the recommendation library) based on a given member profile by inherently measuring the impact of closing the care gap for a member with a similar member profile via determining the reduction of one or more key metrics over a temporal period after closing the care gap. Some example key metrics include a reduction of In-Patient (IP) or Emergency Room (ER) visits in the next 6 months after closing the care gap.

The care recommendation engine 183 may identify the personalized and prioritized recommendations 214 based on the heterogeneous treatment effects 213.

The system 100 may provide model improvement 215 based on feedback (e.g., feedback 159 described with reference to FIG. 1) provided by a care manager in association with the care recommendations 210. For example, model improvement 215 may include testing and learning, implemented by the system 100, that supports iterative improvement of subsequent recommendation results (e.g., subsequent care recommendations 210) generated by the care recommendation engine 183 based on the feedback.

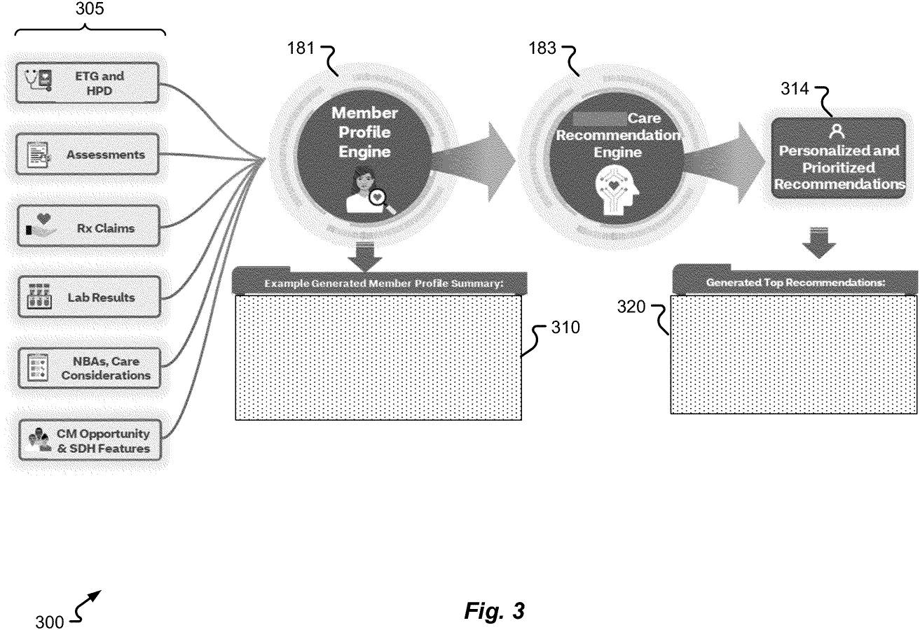

FIG. 3 illustrates an example 300 supportive of providing a care plan in accordance with aspects of the present disclosure. Example 300 may be implemented by the system 100 (e.g., member profile engine 181, care recommendation engine 183, etc.) of FIG. 1. Features of example 300 include aspects of like elements described herein.

The member profile engine 181 may create a member profile (e.g., member profile 211 of FIG. 2) from data 305 provided by various data sources. The data 305 may include historical and available data associated with a member(s).

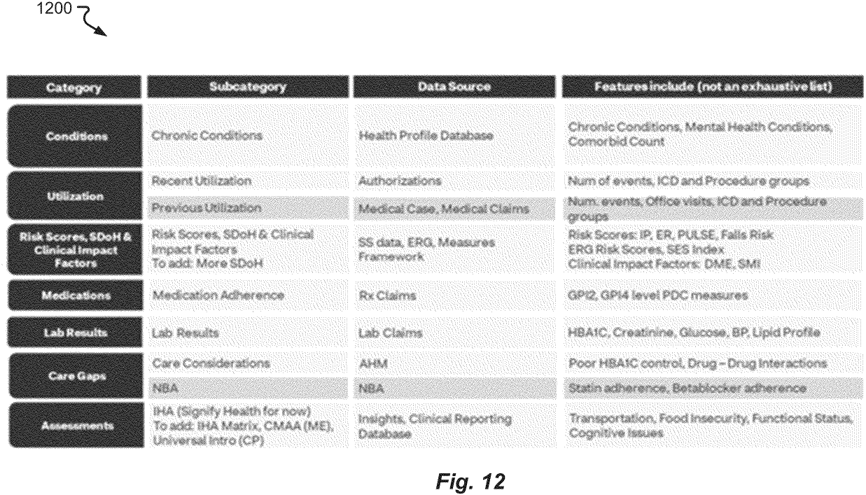

In an example, the data 305 may include, and is not limited to, example information (e.g., input data providable to the member profile engine 181) shown in table 1200 of FIG. 12

In an example, the data 305 may include tele-home assessments and/or in-home assessments. The assessments may be performed by, for example, a vendor. The assessment(s) may include, for example, clinical behavior, clinical status, social determinants, and/or conditions of a patient.

In an example, the member profile engine 181 may generate a profile summary 310 based on the data 305. The profile summary 310 may be separate from or included in the member profile.

In an example, the profile summary 310 may include the following details for a member:

Overview: 19-year-old. Female. Member uses 5 or more drugs (polypharmacy). Member’s Risk Triggers: High Inpatient (IP) Admission Risk. High-Cost Risk. High emergency room (ER) Risk. High Demographic Risk.

Member’s Clinical impact Factors: Medication Non-Adherence. Durable Medical Equipment (DME) Significant. Member is non-adherent to the following Medications in the last 6 months: INSULIN. Member has the following Lab Results in the last 6 months: HBA1c Greater than 9.

In the last 12 months, Member has episodes related to the following: Migraine headache Member’s Previous Utilization (Claims/Authorizations): Migraine/Other Headaches. Acute Admission Type.

The care recommendation engine 183 may generate personalized and prioritized recommendations 314 based on the member profile (or the profile summary 310) and a recommendation library described herein (e.g., recommendation library 152, recommendation library 212).

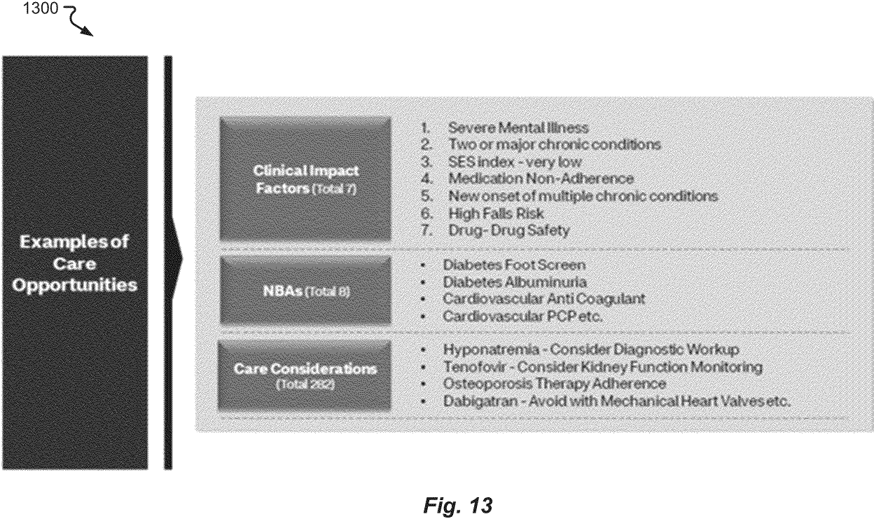

In an example, the recommendation library may include, but is not limited to, example information (e.g., care recommendations, care opportunities) shown in table 1300 of FIG. 13

The care recommendation engine 183 may generate a set of top recommendations 320 from the personalized and prioritized recommendations 314. For example, the set of top recommendations 320 may be a subset of the personalized and prioritized recommendations 314.

In an example, the set of top recommendations 320 may include the following recommendations:

1. Diabetes - lipoprotein cholesterol (LDL-C) control.

2. Consider screening for high blood pressure and follow-up documented.

3. Consider Tdap vaccine.

4. BMI screening and follow-up.

5. Consider weight.

Accordingly, for example, the system 100 supports leveraging holistic member information to recommend personalized and prioritized recommendations 314 (e.g., personalized and prioritized opportunities). For example, the care recommendation engine 183 may gain an understanding of the member (e.g., regarding risk triggers, clinical impact factors, previous healthcare utilization, etc.) based on the profile summary 310. Additionally, or alternatively, the system 100 may support providing the profile summary 310 to the care manager for review. In another example, the system 100 may provide the set of top recommendations 320 (e.g., in a report, for example, in an electronic communication 155 of FIG. 1) to the care manager, based on which the care manager may identify areas of concern for the member and highly impactable (e.g., valuable) opportunities for the member. Additionally or alternatively, the system 100 may support providing top recommendations and context for the top recommendations such that the care manager does not need to analyze why a recommendation was provided.

FIGS. 4A through 4F illustrates examples supportive of providing a care plan in accordance with aspects of the present disclosure. The examples include aspects of member profile generation and ranking techniques implemented by the system 100 (e.g., member profile engine 181, care recommendation engine 183) that support providing clinically guided, analytically optimized care recommendations. Features of FIGS. 4A through 4F include aspects of like elements described herein.

FIG. 4A illustrates an example of the care recommendation engine 183 checking the eligibility of a member 405 with respect to available opportunities 453 included in an opportunity library 452. The opportunity library 452 may include aspects of a recommendation library 152 of FIG. 1 or a recommendation library 212 of FIG. 2.

For example, at 415, the care recommendation engine 183 determines whether the member 405 is eligible for an opportunity 453-a (e.g., Track Asthma Peak Flow) from the opportunity library 452. In an example, the care recommendation engine 183 determines the member 405 is not eligible for the opportunity 453-a. Since the member 405 is not eligible, the care recommendation engine 183 may determine whether the member 405 is eligible for the next available opportunity 453 (e.g., opportunity 453-b, adult BMI assessment).

The care recommendation engine 183 may maintain a table 410 of opportunities 453 for which the member 405 has been checked for eligibility. For each of the opportunities 453, the table 410 may include an indication (e.g., a numerical indication) of a corresponding treatment effect and an indication (e.g., yes, no) of whether the opportunity 453 is statistically significant. Examples of the table 410 and the included indications are later described with reference to FIGS. 4C through 4E.

Referring to FIG. 4B, the care recommendation engine 183 determines whether the member 405 is eligible for opportunity 453-b (e.g., adult BMI assessment).

In an example, the care recommendation engine 183 determines the member 405 is eligible for the opportunity 453-b. Since the member 405 is eligible, then at 420, the care recommendation engine 183 determines whether the opportunity 453-b is still open for the member 405. In the example, the care recommendation engine 183 determines the opportunity 453-b is no longer available, and the care recommendation engine 183 may determine whether the member 405 is eligible for the next available opportunity 453 (e.g., opportunity 453-c, HBA1C Monitoring).

Referring to FIG. 4C, the care recommendation engine 183 determines whether the member 405 is eligible for opportunity 453-c (e.g., HBA1C Monitoring).

In an example, the care recommendation engine 183 determines the member 405 is eligible for the opportunity 453-c. Since the member 405 is eligible, then at 420, the care recommendation engine 183 determines whether the opportunity 453-c is still open for the member 405. In the example, the care recommendation engine 183 determines the opportunity 453-c is still available for the member 405.

At 425, the care recommendation engine 183 may identify other members 426 (e.g., member 426-a through member 426-c) who are similar to the member 405. For example, the care recommendation engine 183 identifies that member 426-a (e.g., Jane), member 426-b (e.g., Kim), and member 426-c (e.g., Larisa) are similar to the member 405. In an example, member 405 and the other members 426 may have profile summaries (e.g., profile summaries 310 described with reference to FIG. 3) that are similar with respect to one or more criteria (e.g., overview, member’s risk triggers, member’s clinical impact factors, member’s previous utilization (claims/authorizations), etc.).

At 430, the care recommendation engine 183 may identify members 426 for which the opportunity 453-c (e.g., HBA1C Monitoring) has been addressed. For example, the care recommendation engine 183 identifies that the opportunity 453-c has been addressed for member 426-a and member 426-b.

At 435, the care recommendation engine 183 may perform a lookback (e.g., a member lookback) to determine whether, within a predetermined amount of time (e.g., 6 months), the number of inpatient visits (also referred to herein as inpatient admissions) decreased for any of the members 1026. For example, the care recommendation engine 183 identifies that the number of inpatient visits decreased by 2 for member 426-a, the number of inpatient visits decreased by 1 for member 426-b, and the number of inpatient visits did not change for member 426-c.

At 440, the care recommendation engine 183 determines a treatment effect associated with the members 426 (e.g., member 426-a, member 426-b) for which the opportunity 453-c was addressed. For example, the care recommendation engine 183 calculates a difference in average reduction in number of inpatient admissions for member 426-a and member 426-b (for whom opportunity 453-c was addressed) versus average reduction in number of inpatient admissions for member 426-c (for whom opportunity 453-c was not addressed). In an example, the care recommendation engine 183 determines (e.g., calculates) a treatment effect of 1.5.

At 445, the care recommendation engine 183 determines whether the treatment effect is significant (e.g., based on a comparison of the treatment effect value to a threshold value).

FIG. 4D illustrates an example of the table 410. Based on the data included in the table 410, the care recommendation engine 183 may provide care recommendations based on a likelihood of the member 405 to close care gaps associated with the opportunities 453. For example, the care recommendation engine 183 may select an opportunity 453 (e.g., opportunity 453-c and opportunity 453-d) based on whether the member 405 has eligibility for the opportunity 453, whether the opportunity 453 is open for the member 405, whether a treatment effect associated with the opportunity 453 satisfies a criteria, and/or whether the opportunity 453 is statistically significant.

Referring to FIG. 4E, the care recommendation engine 183 may assign treatment effect ranks based on opportunity value. For example, referring to table 410, the care recommendation engine 183 may assign prioritized ranks (also referred to herein as an opportunity rank) to the opportunity 453-c and opportunity 453-d based on respective opportunity values.

For example, referring to table 460, the care recommendation engine 183 may calculate an opportunity value of 1.2 for opportunity 453-c and an opportunity value of 0.9 for opportunity 453-d. The care recommendation engine 183 may assign an opportunity rank of ‘1’ to opportunity 453-c and assign an opportunity rank of ‘2’ to opportunity 453-d.

Referring to table 470 of FIG. 4F, for generated opportunities 453 (e.g., opportunity 453-f through opportunity 453-i) having an assigned treatment effect rank, the care recommendation engine 183 may further sort the opportunities 453, first by clinical prioritization rank, followed by treatment effect rank. Based on the sorting, the care recommendation engine 183 may provide a final opportunity rank (e.g., illustrated at table 480) for the generated opportunities.

The care recommendation engine 183 may provide the opportunities 453 (e.g., opportunity 453-f through opportunity 453-i) based on respective final opportunity ranks.

For example, the care recommendation engine 183 may provide recommended action(s) 457 associated with addressing the opportunities 453. For example, the recommended action(s) 457 may include an indication of the opportunities 453 and respective final opportunity ranks. The recommended action(s) 457 may be examples of recommended action(s) 157 described with reference to FIG. 1.

Accordingly, for example, the system 100 may support providing clinically guided, and analytically optimized care recommendations that are specific, measurable, actionable, reportable, and time bound.

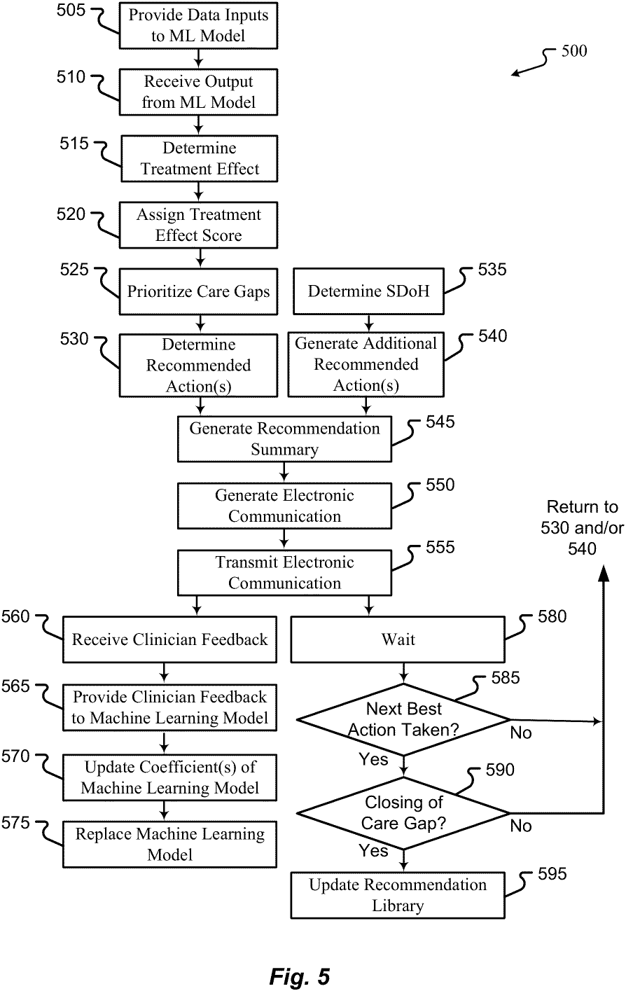

FIG. 5 illustrates an example of a process flow 500 that supports providing care recommendations in accordance with aspects of the present disclosure. For example, the process flow 500 may support automatically generating a patient care plan in accordance with aspects of the present disclosure. In some examples, process flow 500 may implement aspects of the communication device 105 or the server 135 described with reference to FIG. 1.

In the following description of the process flow 500, the operations may be performed in a different order than the order shown, or the operations may be performed in different orders or at different times. Certain operations may also be left out of the process flow 500, or other operations may be added to the process flow 500.

It is to be understood that while a device 105 and/or a server 135 is described as performing a number of the operations of process flow 500, any device (e.g., another communication device 105 in communication with the communication device 105 or the server 135, another server 135 in communication with the communication device 105 or the server 135, etc.) may perform the operations shown.

At 505, the process flow 500 may include providing data inputs to a machine learning model. In some aspects, the data inputs may include electronic patient data obtained from a plurality of electronic records describing a health history of the patient.

In some aspects, the electronic patient data may include claims-based electronic data.

In some aspects, the electronic patient data further may include electronic medical record (EMR) data.

In some aspects, the claims-based electronic data may include data describing at least one insurance medical and/or insurance claim made by at least one of the patient and a healthcare provider of the patient.

In some aspects, the electronic patient data may include device data obtained from at least one device associated with the patient.

At 510, the process flow 500 may include receiving an output from the machine learning model. In some aspects, the output received from the machine learning model is generated based on the machine learning model processing the data inputs and includes a plurality of identified care gaps for the patient.

At 515, the process flow 500 may include determining a treatment effect for each of the plurality of identified care gaps.

At 520, the process flow 500 may include, based on the treatment effect determined for each of the plurality of identified care gaps, assigning a treatment effect score to each of the plurality of identified care gaps.

In some aspects, the treatment effect score for each identified care gap is based, at least in part, on a prediction of success associated with closing each identified care gap.

At 525, the process flow 500 may include prioritizing the plurality of identified care gaps based on the treatment effect score assigned thereto.

At 530, the process flow 500 may include, based on the prioritization of the plurality of identified care gaps, determining one or more recommended patient actions (e.g., recommended action(s) 157 of FIG. 1) for the patient.

In some aspects, the one or more recommended patient actions are determined, at least in part, with reference to a patient library that includes a plurality of electronic patient data records with care gap information and information describing a success of closing a care gap with an action.

In some aspects, the information describing the success of closing the care gap with the action may include a count of a number of patient admissions to a healthcare facility following an identification of the care gap.

At 535, the process flow 500 may include determining a social determinant of health (SDoH) for the patient.

At 540, the process flow 500 may include generating at least one additional recommended patient action (e.g., SDoH recommendation 156 of FIG. 1) for the patient based on the determined SDoH for the patient. In some aspects, the at least one additional recommended patient action for the patient provides a description of an action for a clinician to address a barrier for the patient using an SDoH resource.

At 545, the process flow 500 may include automatically generating a recommendation summary (e.g., recommendation summary 158 of FIG. 1) that includes a summarized description of the one or more recommended patient actions for the patient. In some aspects, the recommendation summary includes an additional summarized description of the at least one additional recommended patient action.

In some aspects, the recommendation summary is generated, at least in part, with a natural language generation (NLG) model.

At 550, the process flow 500 may include generating an electronic communication (e.g., communication 155 described with reference to FIG. 1) that describes the one or more recommended patient actions for the patient. In some aspects, the electronic communication describes the at least one additional recommended patient action.

In some examples, the process flow 500 may include including the recommendation summary in the electronic communication.

In some aspects, the one or more recommended patient actions for the patient are generated, at least in part, using a heterogeneous treatment effect (HTE) model. In some aspects, the HTE model is updated using a reinforcement learning-based formulation that dynamically updates the HTE model based on clinician feedback while the HTE model is being used to generate the one or more recommended patient actions.

In some aspects, the one or more recommended patient actions may include a description of where to send the patient to address a care gap from the plurality of identified care gaps.

In some aspects, the one or more recommended patient actions may include a next best action for the patient to take in connection with closing a care gap from the plurality of identified care gaps. In some aspects, the next best action is associated with closing more than one care gap from the plurality of identified care gaps. In some aspects, the next best action corresponds to an action that is predicted most likely to be taken by the patient. In some aspects, the next best action corresponds to at least one of the following: a visit to a healthcare provider, a change in a medical treatment, a change in a prescription, a change in diet, a change in activity, a blood test, and a medical examination.

At 555, the process flow 500 may include transmitting the electronic communication via a communication network (e.g., communication network 140) to a communication device.

In some aspects, the electronic communication is transmitted to a communication device of the patient. In some aspects, the electronic communication is transmitted to a communication device of a care manager of the patient. In some aspects, the electronic communication is transmitted via a selected communication channel.

In some aspects, the selected communication channel is selected based on a probability of closing a care gap having the highest treatment effect score and in some aspects, the selected communication channel may include at least one of email, direct mail, SMS, and an automated outbound calling campaign.

At 560, the process flow 500 may include receiving clinician feedback (e.g., feedback 159 described with reference to FIG. 1) for the one or more recommended patient actions.

At 565, the process flow 500 may include providing the clinician feedback as training data to the machine learning model.

At 570, the process flow 500 may include updating at least one coefficient of the machine learning model based on providing the training data to the machine learning model.

At 575, the process flow 500 may include replacing the machine learning model with an updated version of the machine learning model. In some aspects, the updated version of the machine learning model may include the updated at least one coefficient.

Additionally, or alternatively, at 580, the process flow 500 may include waiting a predetermined amount of time after transmitting the electronic communication.

For example, at 585, the process flow 500 may include after the predetermined amount of time, performing a patient lookback to determine whether the patient took the next best action within the predetermined amount of time.

In another example, at 590, the process flow 500 may include, if the patient took the next best action within the predetermined amount of time, determining whether the next best action resulted in a partial or complete closing of the care gap.

At 595, the process flow 500 may include updating a recommendation library based on whether the next best action results in a partial or complete closing of the care gap.

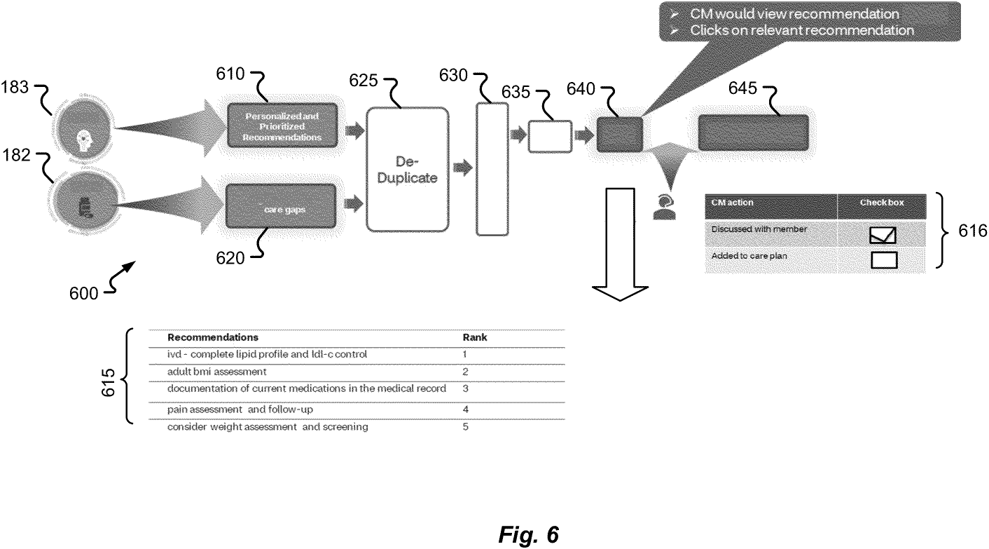

FIG. 6 illustrates aspects of an example process flow 600 that supports providing a care plan in accordance with aspects of the present disclosure. In some examples, process flow 600 may be implemented by the system 100 (e.g., communication device 105, server 135, care gap management engine 182, care recommendation engine 183) in association with tracking care recommendations with respect to comprehensive end-stage renal disease (ESRD) care (CEC). Features of process flow 600 include aspects of like elements described herein, and descriptions of like elements are omitted for brevity.

The care recommendation engine 183 may provide personalized and prioritized recommendations 610 (e.g., recommended action(s) 157, recommended action(s) 457, etc.).

The care gap management engine 182 may provide care gap data 620. Care gap data 620 may include identified care gaps for members as described herein.

At 625, the process flow 600 may include performing de-duplication operations. The de-duplication operations may include operations associated with removing duplicate data among the prioritized recommendations 610 and care gap data 620.

At 630, the process flow 600 may include an opportunity production launchpad.

At 635, the process flow 600 may include an opportunity router.

At 640, the process flow 600 may include a review of care recommendations provided at 635. In an example, communication device 105 may display care recommendations via user interface 130. A care manager may view and select between the care recommendations. At 640, the process flow 600 may include a member information console.

At 645, the process flow 600 may include a care planning console.

For example, as illustrated at example user interface 615 and user interface 616, the care manager may select a care recommendation and indicate whether an action (e.g., discussed with member, added to care plan, etc.) has been completed with respect to the care recommendation. The inputs by the care manager may be an example of feedback 159 described with reference to FIG. 1.

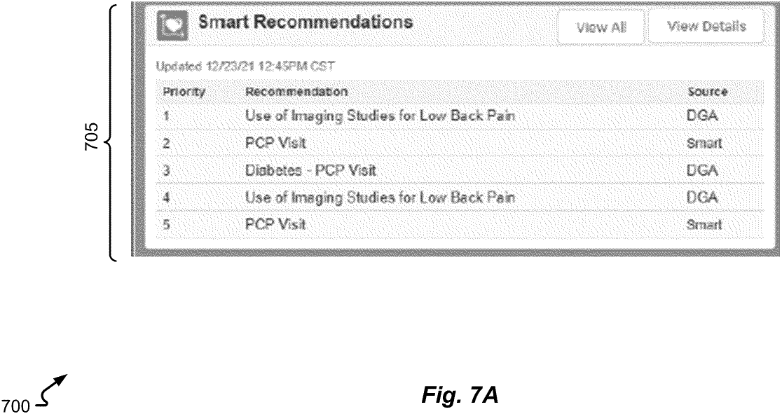

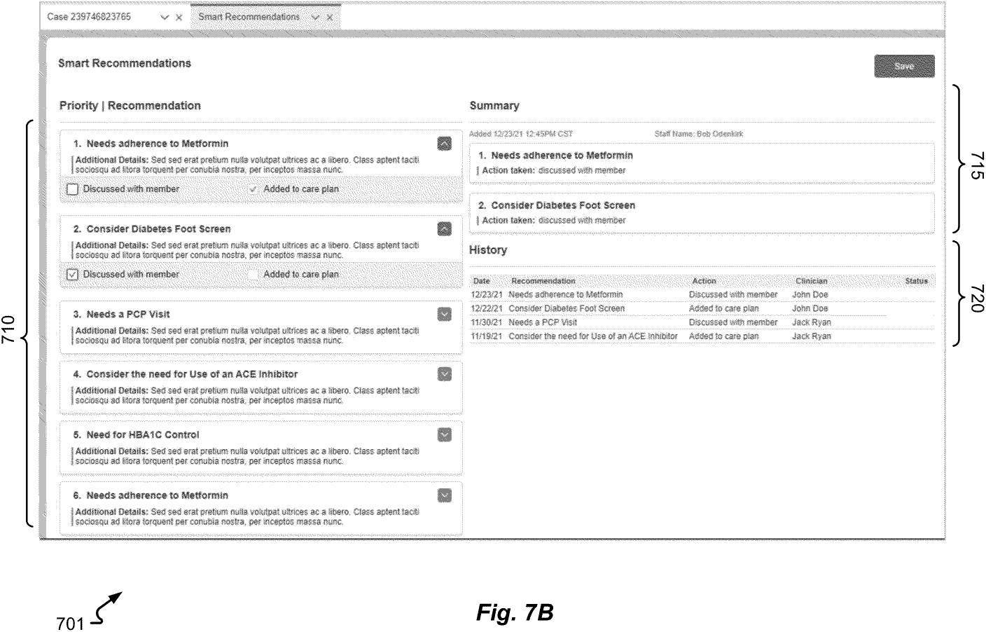

FIG. 7A illustrates an example dashboard 700 that supports providing a care plan in accordance with aspects of the present disclosure. FIG. 7B illustrates an example action tracking view 701 that supports providing a care plan in accordance with aspects of the present disclosure.

In some examples, the dashboard 700 and action tracking view 701 may be implemented by the system 100 (e.g., communication device 105) in association with providing and managing a care plan. Features of the dashboard 700 and action tracking view 701 include aspects of like elements described herein.

Referring to FIG. 7A, the system 100 may display a list 705 of care recommendations on the dashboard 700. The list 705 of care recommendations may include priority information and source information associated with each care recommendation. Based on a user input selecting the list 705, the system 100 may display the action tracking view 701.

Referring to FIG. 7B, the action tracking view 701 may include a list 710 of care recommendations, a recommendation summary 715, and a history 720.

The action tracking view 701 (e.g., list 710) may support user inputs for indicating actions taken (e.g., discussed with member, added to care plan, etc.) by a user with respect to each care recommendation. In some aspects, for each care recommendation, the list 710 may include information of available medical centers (e.g., hospitals, clinics, pharmacies, etc.) that provide treatment associated with the care recommendation. In an example, the action tracking view 701 (e.g., the list 710) may include location information of available medical centers based on distance from a location (e.g., current location, home address, etc.) associated with the member.

The recommendation summary 715 may include an indication of top (e.g., highest priority) care recommendations from the list 710. In an example, recommendation summary 715 may include location information of available medical centers associated with the top care recommendations. The history 720 may include descriptions of previous care recommendations and associated information (e.g., date, actions taken, clinician, status, etc.).

In some aspects, a user (e.g., healthcare personnel, a care manager, etc.) may view the list 710 of care recommendations and actively track any care recommendations discussed with a member. In some examples, the action tracking view 701 may support documenting (e.g., in real-time, in near real-time) histories of tracked care recommendations.

Aspects of the present disclosure support generating “assessment data” to show responses to questions (e.g., whether a care recommendation has been discussed with a member and/or added to a care plan). In some aspects, the system 100 may support providing the assessment data as feedback 159 in association with training a machine learning model(s) 184. For example, the assessment data may be implemented as a ground truth dataset.

In some aspects, user interactions (e.g., personnel interactions, care manager interactions, healthcare provider interactions, etc.) with the dashboard 700 and/or the action tracking view 701 may be incorporated for training the machine learning model(s) 184 described herein. For example, based on the user interactions with the dashboard 700 and/or the action tracking view 701, the system 100 (e.g., communication device 105, server 135) may generate new feedback (e.g., feedback 159) for model training.

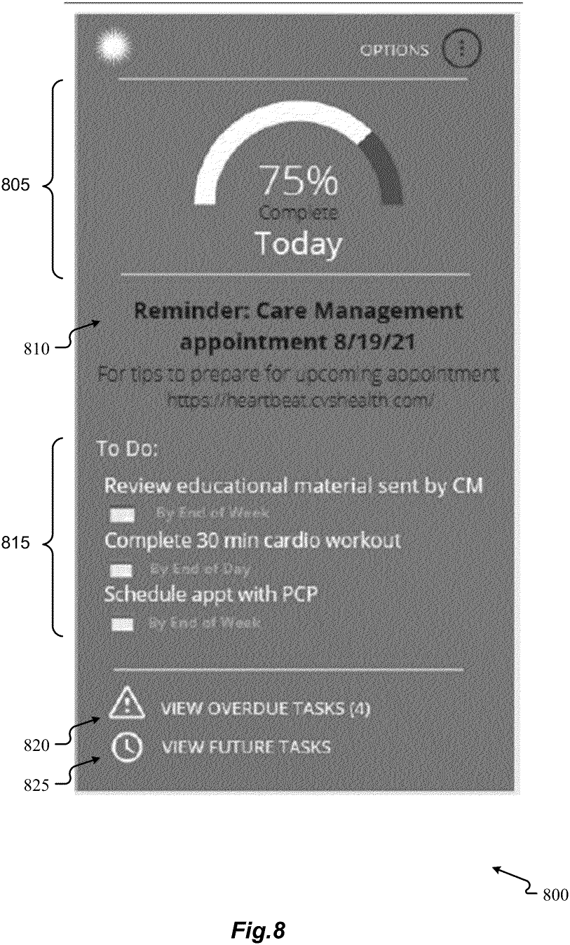

FIG. 8 illustrates an example user interface 800 that supports providing a care plan in accordance with aspects of the present disclosure.

In some examples, the user interface 800 may be implemented by the system 100 (e.g., communication device 105) in association with member adherence to a care plan. The user interface 800 may be displayed, for example, at a communication device 105 of a member (e.g., via an application of the communication device 105, via the browser 125, etc.).

The user interface 800 may include a completion indicator 805, reminder notifications 810, a list 815 of care recommendations assigned to the member, an overdue notification icon 820, and a future task icon 825.

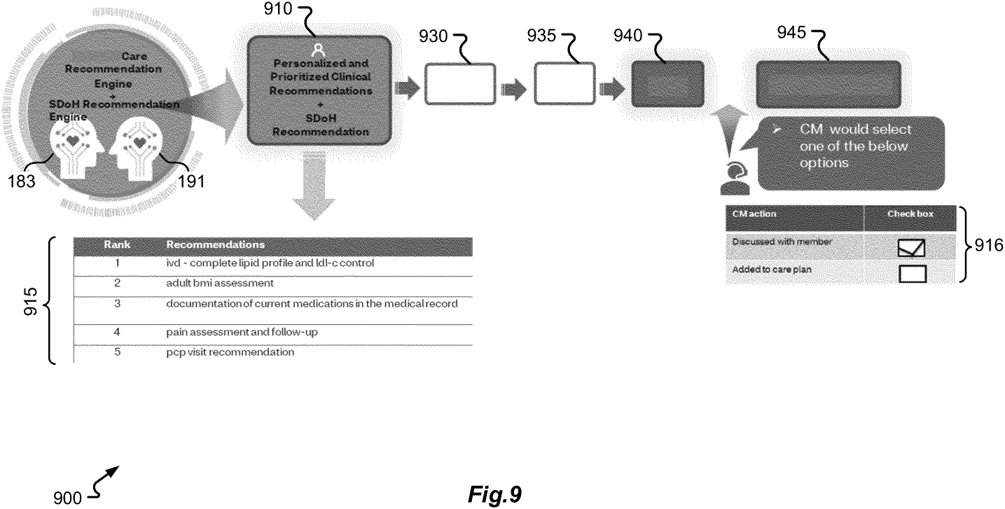

FIG. 9 illustrates aspects of an example process flow 900 that supports providing a care plan in accordance with aspects of the present disclosure. In some examples, process flow 900 may be implemented by the system 100 (e.g., communication device 105, server 135, care gap management engine 182, care recommendation engine 183, SDoH recommendation engine 191) in association with tracking care recommendations with respect to CEC.

The process flow 900 includes aspects of integrating aspects of care recommendations provided by care recommendation engine 183 and SDoH recommendations provided by SDoH recommendation engine 191. Features of process flow 900 include aspects of like elements described herein, for example, with respect to process flow 600 of FIG. 6. Detailed descriptions of similar elements are omitted for brevity.

The care recommendation engine 183 and the SDoH recommendation engine 191 may provide care recommendations 910. Care recommendations 910 may include a combination of personalized and prioritized clinical recommendations (e.g., recommended action(s) 157 provided by the care recommendation engine 183) and SDoH recommendations (e.g., SDoH recommendation(s) 156 provided by the SDoH recommendation engine 191).

At 925, the process flow 900 may include performing de-duplication operations. The de-duplication operations may include operations associated with removing duplicate data among the prioritized recommendations 910 and care gap data 920.

At 930, the process flow 900 may include an opportunity production launchpad.

At 935, the process flow 900 may include an opportunity router.

At 940, the process flow 900 may include a review of care recommendations provided at 935. In an example, communication device 105 may display care recommendations via user interface 130. A care manager may view and select between the care recommendations. At 940, the process flow 900 may include a member information console.

At 945, the process flow 900 may include a care planning console.

For example, as illustrated at example user interface 915 and user interface 916, the care manager may select a care recommendation and indicate whether an action (e.g., discussed with member, added to care plan, etc.) has been completed with respect to the care recommendation. The inputs by the care manager may be an example of feedback 159 described with reference to FIG. 1.

In some aspects, the care recommendations 910 (displayed at user interface 915) may include an indication of SDoH recommendations (e.g., member may need transportation support), in addition to care recommendations.

FIGS. 10A and 10B illustrates examples 1000 and 1001 supportive of providing a care plan in accordance with aspects of the present disclosure. Examples 1000 and 1001 include aspects of an SDoH recommendation algorithm implemented by the system 100 (e.g., SDoH recommendation engine 191) that support providing SDoH recommendations based on identified SDoH barriers. The system 100 supports implementing the SDoH recommendation algorithm by SDoH recommendation engine 191, in combination with aspects of the care recommendation engine 183 described herein. Additionally, or alternatively, aspects of examples 1000 and 1001support implementations of the SDoH recommendation algorithm by the SDoH recommendation engine 191, without the care recommendation engine 183.

Examples 1000 and 1001 may be implemented by the system 100 (e.g., member profile engine 181, care recommendation engine 183, SDoH recommendation engine 191, etc.) of FIG. 1. Features of examples 1000 and 1001 include aspects of like elements described herein, for example, with respect to FIGS. 4A through 4D.

FIG. 10A illustrates an example of the SDoH recommendation engine 191 checking the eligibility of a member 1005 with respect to available opportunities 1053 (e.g., opportunities 1053-a through opportunities 1053-d) included in an opportunity library 1052. For example, the SDoH recommendation engine 191 has determined the member 1005 is eligible for opportunity 1053-c (e.g., HBA1C Monitoring).

The SDoH recommendation engine 191 may maintain a table 1010 of opportunities 1053 for which the member 1005 has been checked for eligibility. For each of the opportunities 1053, the table 1010 may include an indication of whether an SDoH barrier exists in association with the opportunity 1053, an indication (e.g., a numerical indication) of a corresponding treatment effect, and an indication (e.g., yes, no) of whether the opportunity 1053 is statistically significant. Examples of the table 1010 and the included indications are later described with reference to FIG. 10B.

In an example, at 1015, the SDoH recommendation engine 191 determines the member 1005 is eligible for the opportunity 1053-c.

At 1020, the SDoH recommendation engine 191 determines whether the opportunity 1053-c is still open for the member 1005. In the example, the SDoH recommendation engine 191 determines the opportunity 1053-c is still available for the member 1005.

At 1025, the SDoH recommendation engine 191 may identify other members 1026 (e.g., member 1026-a through member 1026-b) who are similar to the member 1005. For example, the care recommendation engine 183 identifies that member 1026-a (e.g., Jane), member 1026-b (e.g., Kim), and member 1026-c (e.g., Larisa) are similar to the member 1005. In an example, member 1005 and the other members 1026 may have profile summaries (e.g., profile summaries 310 described with reference to FIG. 3) that are similar with respect to one or more criteria (e.g., overview, member’s risk triggers, member’s clinical impact factors, member’s previous utilization (claims/authorizations), etc.).

At 1030, the SDoH recommendation engine 191 may identify members 1026 for which an SDoH barrier exists. For example, the SDoH recommendation engine 191 identifies that an SDoH barrier does not exist for member 1026-a and member 1026-b, and that an SDoH barrier exists for member 1026-c.

At 1035, the SDoH recommendation engine 191 may perform a lookback (e.g., a member lookback) to determine whether, within a predetermined amount of time (e.g., 6 months), any of the members 1026 took the opportunity 1053-c (e.g., followed the care recommendation). In some aspects, at 1035, for a member 1026 identified as having taken the opportunity 1053-c within the predetermined amount of time, the care recommendation engine 183 may determine whether taking the opportunity 1053-c resulted in a partial or complete closing of a care gap.

For example, the SDoH recommendation engine 191 identifies that a care gap was closed for member 1026-a and member 1026-b, for which no SDoH barrier exists. The care recommendation engine 183 identifies that a care gap was not closed for member 1026-c, for which an SDoH barrier does exist.

At 1040, the SDoH recommendation engine 191 determines a treatment effect associated with the members 1026. For example, the SDoH recommendation engine 191 calculates a difference in care gap closure for member 1026-a and member 1026-b (who do not have an SDoH barrier(s)) versus care gap closure for member 1026-c (who does have an SDoH barrier(s)). In an example, the SDoH recommendation engine 191 determines (e.g., calculates) a treatment effect of 1.

At 1045, the SDoH recommendation engine 191 determines whether the treatment effect is statistically significant (e.g., based on a comparison of the treatment effect value to a threshold value).

FIG. 10B illustrates an example of the table 1010. Based on the data included in the table 1010, the SDoH recommendation engine 191 may provide SDoH recommendations (e.g., SDoH recommendations 156) linked to care recommendations (e.g., opportunities 1053, also referred to herein as recommended actions 157) provided by the care recommendation engine 183. For example, the SDoH recommendation engine 191 may provide an SDoH recommendation that, for opportunity 1053-c, the member 1005 may need social and financial support in order to take the opportunity 1053-c (e.g., complete the care recommendation).

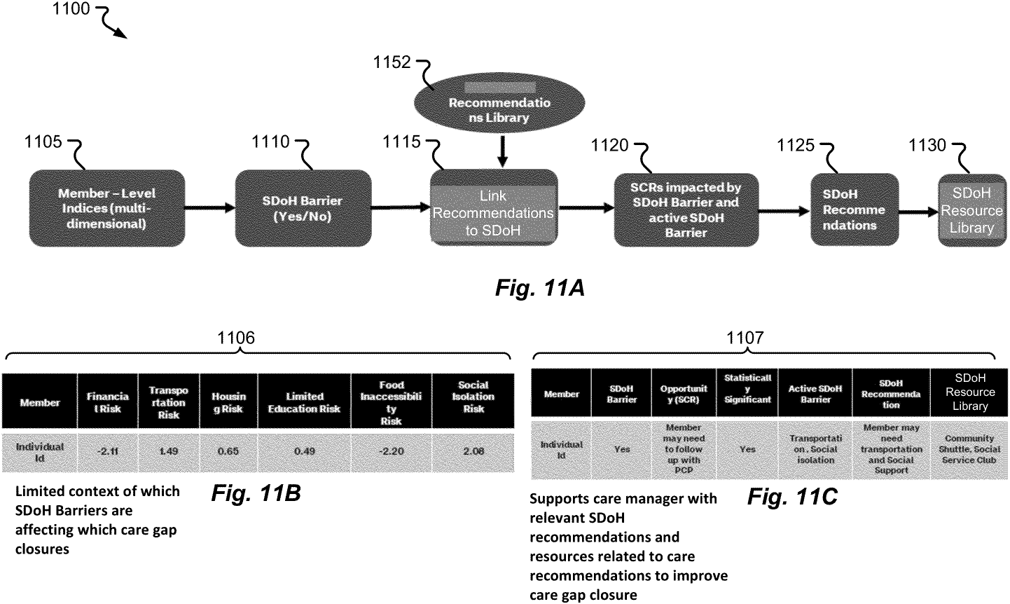

FIGS. 11A through 11C illustrate an example process flow 1100, an example table 1135, and an example table 1140 that support leveraging member level SDoH indices for SDoH recommendations according to aspects of the present disclosure. In an example, the process flow 1100 may be implemented by aspects of the system 100 (e.g., care recommendation engine 183, SDoH recommendation engine 191) described herein. In an example, the process flow 1100 may include aspects of FIGS. 10A and 10B.

At 1105, the process flow 1100 may include determining member level domains/indices from multiple data sources. Data sources may include, for example, public datasets (e.g., census tract level, state and metropolitan area level, county level, etc.), non-public datasets (e.g., at the individual level, for example, self-reported assessments, clinical notes, etc.) and the like, but are not limited thereto.

An example set of domains/indices is illustrated at 1106. Another example set of domains/indices includes: health infrastructure, education, economic condition, health access, food access, employment, social isolation, water, air, housing quality, disability, transport availability, crime, and health habits. Another example set of domains/indices includes: income, poverty, diversity, disability, education, physical activity, family structure, public transportation, employment, and food access. An example set of domains/indices at an individual level include: social isolation, financial risk, housing instability, food inaccessibility, limited education, and limited transportation.

At 1110, the process flow 1100 may include determining whether an SDoH barrier exists for a member(s). In an example, determining whether the SDoH barrier exists may be via population segmentation with respect to risk and impactability.

At 1115, the process flow 1100 may include linking care recommendations (e.g., from recommendation library 1152) to SDoH. For example, at 1115, the process flow 1100 may include analyzing the impact of / addressing SDoH barriers on clinical care gaps closure (e.g., care recommendations).

At 1120, the process flow 1100 may include identifying care recommendations impacted by SDoH barrier and active SDoH barrier. For example, at 1120, the process flow 1100 may include identifying risky SDoH barriers for care recommendations where a difference in care gap closure (e.g., care recommendations) is statistically significant.

At 1125, the process flow 1100 may include generating and providing SDoH recommendations for improving care gap closure rate. An example of SDoH recommendations is illustrated at 1107. The process flow 100 may include storing the SDoH recommendations to an SDoH resource library at 1130.

The process flow 1100 may include providing care recommendations based on the SDoH recommendations.

Aspects of the present disclosure may be applied to various types of healthcare organizations.

A number of implementations have been described. Nevertheless, it will be understood that additional modifications may be made without departing from the scope of the inventive concepts described herein, and, accordingly, other examples are within the scope of the following claims.

Various example aspects described herein may be applied to various types of healthcare organizations. Aspects of the present disclosure may be applied to supporting providers with member health summaries towards obtaining better health outcomes for the members through enabling access to healthcare via provider partners, examples of which are described in Exhibit A.

Additional details of the disclosure are in the following attachment(s), each of which is incorporated herein by this reference:

### Exhibit A - Provider Partner Case Round Summaries_IP

The exemplary systems and methods of this disclosure have been described in relation to examples of a communication device 105 and a server 135. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the examples illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed examples, configuration, and aspects.

A number of variations and modifications of the disclosure can be used. It would be possible to provide for some features of the disclosure without providing others.

In yet another example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another examples, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another example, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the examples with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various examples, configurations, and aspects, includes components, methods, processes, systems and/or apparatus substantially as depicted and described herein, including various examples, subcombinations, and subsets thereof. Those of skill in the art will understand how to make and use the systems and methods disclosed herein after understanding the present disclosure. The present disclosure, in various examples, configurations, and aspects, includes providing devices and processes in the absence of items not depicted and/or described herein or in various examples, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more examples, configurations, or aspects for the purpose of streamlining the disclosure. The features of the examples, configurations, or aspects of the disclosure may be combined in alternate examples, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed example, configuration, or aspect. Thus, the following claims are hereby incorporated into this Detailed Description, with each claim standing on its own as a separate preferred example of the disclosure.

Moreover, though the description of the disclosure has included description of one or more examples, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative examples, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an example that is entirely hardware, an example that is entirely software (including firmware, resident software, microcode, etc.) or an example combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A method of automatically generating a patient care plan, the method comprising:
providing data inputs to a machine learning model, wherein the data inputs comprise electronic patient data obtained from a plurality of electronic records describing a health history of the patient;
receiving an output from the machine learning model, wherein the output received from the machine learning model is generated based on the machine learning model processing the data inputs and includes a plurality of identified care gaps for the patient;
determining a treatment effect for each of the plurality of identified care gaps;
based on the treatment effect determined for each of the plurality of identified care gaps, assigning a treatment effect score to each of the plurality of identified care gaps;
prioritizing the plurality of identified care gaps based on the treatment effect score assigned thereto;
based on the prioritization of the plurality of identified care gaps, determining one or more recommended patient actions for the patient;
generating an electronic communication that describes the one or more recommended patient actions for the patient; and
transmitting the electronic communication via a communication network to a communication device.

1. The method of claim 1, wherein the electronic communication is transmitted to a communication device of the patient.

2. The method of claim 1, wherein the electronic communication is transmitted to a communication device of a care manager of the patient.

3. The method of claim 1, wherein the one or more recommended patient actions are determined, at least in part, with reference to a patient library that includes a plurality of electronic patient data records with care gap information and information describing a success of closing a care gap with an action.

4. The method of claim 4, wherein the information describing the success of closing the care gap with the action comprises a count of a number of patient admissions to a healthcare facility following an identification of the care gap.

5. The method of claim 1, wherein the treatment effect score for each identified care gap is based, at least in part, on a prediction of success associated with closing each identified care gap.

6. The method of claim 1, wherein the electronic communication is transmitted via a selected communication channel.

7. The method of claim 7, wherein the selected communication channel is selected based on a probability of closing a care gap having the highest treatment effect score and wherein the selected communication channel comprises at least one of email, direct mail, SMS, and an automated outbound calling campaign.

8. The method of claim 1, wherein the electronic patient data comprises claims-based electronic data.

9. The method of claim 9, wherein the electronic patient data further comprises electronic medical record (EMR) data.

10. The method of claim 9, wherein the claims-based electronic data comprises data describing at least one insurance medical and/or insurance claim made by at least one of the patient and a healthcare provider of the patient.

11. The method of claim 1, wherein the electronic patient data comprises device data obtained from at least one device associated with the patient.

12. The method of claim 1, further comprising:
receiving clinician feedback for the one or more recommended patient actions;
providing the clinician feedback as training data to the machine learning model; and
updating at least one coefficient of the machine learning model based on providing the training data to the machine learning model.

13. The method of claim 13, further comprising:
replacing the machine learning model with an updated version of the machine learning model, wherein the updated version of the machine learning model comprises the updated at least one coefficient.

15. The method of claim 1, wherein the one or more recommended patient actions comprise a next best action for the patient to take in connection with closing a care gap from the plurality of identified care gaps.

16. The method of claim 15, wherein the next best action is associated with closing more than one care gap from the plurality of identified care gaps.

17. The method of claim 15, wherein the next best action corresponds to an action that is predicted most likely to be taken by the patient.

18. The method of claim 15, wherein the next best action corresponds to at least one of the following: a visit to a healthcare provider, a change in a medical treatment, a change in a prescription, a change in diet, a change in activity, a blood test, and a medical examination.

19. The method of claim 15, further comprising:
waiting a predetermined amount of time after transmitting the electronic communication;
after the predetermined amount of time, performing a patient lookback to determine whether the patient took the next best action within the predetermined amount of time;
if the patient took the next best action within the predetermined amount of time, determining whether the next best action resulted in a partial or complete closing of the care gap; and
updating a recommendation library based on whether the next best action results in a partial or complete closing of the care gap.

20. The method of claim 1, further comprising:
automatically generating a recommendation summary that includes a summarized description of the one or more recommended patient actions for the patient; and
including the recommendation summary in the electronic communication.

21. The method of claim 20, wherein the recommendation summary is generated, at least in part, with a natural language generation (NLG) model.

22. The method of claim 1, wherein the one or more recommended patient actions for the patient are generated, at least in part, using a heterogeneous treatment effect (HTE) model.

23. The method of claim 22, wherein the HTE model is updated using a reinforcement learning-based formulation that dynamically updates the HTE model based on clinician feedback while the HTE model is being used to generate the one or more recommended patient actions.

24. The method of claim 1, wherein the one or more recommended patient actions comprises a description of where to send the patient to address a care gap from the plurality of identified care gaps.

25. The method of claim 1, further comprising:
determining a social determinant of health (SDoH) for the patient; and
generating at least one additional recommended patient action for the patient based on the determined SDoH for the patient, wherein the at least one additional recommended patient action for the patient provides a description of an action for a clinician to address a barrier for the patient using an SDoH resource,
wherein the electronic communication describes the at least one additional recommended patient action.
